(19) United States
(12) Patent Application Publication
(10) Pub. No.: US 2023/0220088 A1
(43) Pub. Date: Jul. 13, 2023

(54) Anti-CCR5 Monoclonal Antibody-Based Compositions and Methods

(71) Applicant: Maddon Advisors LLC, Scarsdale, NY (US)

(72) Inventor: Paul J. Maddon, Scarsdale, NY (US)

(73) Assignee: Maddon Advisors LLC, Scarsdale, NY (US)

(21) Appl. No.: 18/002,066

(22) PCT Filed: Jun. 22, 2021
(86) PCT No.: PCT/US2021/038349

§ 371 Date: Dec. 16, 2022

Related U.S. Application Data
(60) Provisional application No. 63/042,661, filed on Jun. 23, 2020.
(60) Provisional application No. 63/062,506, filed on Aug. 7, 2020.

(51) Int. Cl. C07K 16/28; C12N 15/86; A61P 31/18

(52) U.S. Cl. CPC C07K 16/2866; C12N 15/86; A61P 31/18; C07K 2317/24; C07K 2317/94; C12N 2750/14143

(57) ABSTRACT

This invention provides humanized IgG4 monoclonal antibodies having the light and heavy chain variable region amino acid sequences of PRO 140, wherein the antibody comprises (i) a heavy chain modification that inhibits half antibody formation, (ii) a heavy chain modification that increases the antibody's terminal half-life, and, optionally, (iii) a heavy chain modification that lowers the antibody's effector function. This invention also provides a related humanized IgG2/IgG4 monoclonal fusion antibody. This invention further provides related nucleic acid molecules; recombinant vectors; recombinant AAV particles; pharmaceutical compositions; prophylactic and therapeutic methods for addressing HIV-1 infection, SARS-CoV-2 infection, and CCR5-mediated disorders; and kits for performing these methods.

This application claims the benefit of U.S. Provisional Application No. 63/042,661, filed Jun. 23, 2020, and U.S. Provisional Application No. 63/062,506, filed Aug. 7, 2020, the contents of both of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to engineered antibodies that target human CCR5, as well as related engineered viruses. These antibodies and viruses are useful for addressing SARS-CoV-2 infection, HIV-1 infection, and other disorders.

### BACKGROUND OF THE INVENTION

Since the beginning of the AIDS epidemic, there has been an ongoing worldwide effort to develop effective anti-HIV-1 therapeutics and prophylactics. To date, this effort has yielded many failures and only a few successes. And, even the successful therapeutics and prophylactics are not without drawbacks such as serious side effects and the need for repeated administration. More recently, since the beginning of the COVID-19 outbreak, there has been—and continues to be—an intensive worldwide effort to develop effective anti-SARS-CoV-2 therapeutics and prophylactics. To date, this nascent effort has not succeeded.

For at least these reasons, there is an unmet need for a superior way to treat and prevent HIV-1 and SARS-CoV-2 infections, as well as other disorders addressable by targeting CCR5.

### SUMMARY OF THE INVENTION

This invention provides a humanized monoclonal antibody having the light chain variable region amino acid sequence set forth in FIG. 1A and the heavy chain variable region amino acid sequence set forth in FIG. 1B or 1C, wherein the antibody comprises a heavy chain modification that increases the antibody's terminal half-life.

This invention also provides a first humanized IgG4 monoclonal antibody having the light chain variable region amino acid sequence set forth in FIG. 1A and the heavy chain variable region amino acid sequence set forth in FIG. 1B or 1C, wherein the antibody comprises (i) a heavy chain modification that inhibits half antibody formation, and (ii) a heavy chain modification that increases the antibody's terminal half-life.

This invention further provides a humanized monoclonal antibody having the light chain variable region amino acid sequence set forth in FIG. 1A and the heavy chain variable region amino acid sequence set forth in FIG. 1B or 1C, wherein the antibody comprises (i) a heavy chain modification that increases the antibody's terminal half-life, and (ii) a heavy chain modification that lowers the antibody's effector function.

This invention further provides a second humanized IgG4 monoclonal antibody having the light chain variable region amino acid sequence set forth in FIG. 1A and the heavy chain variable region amino acid sequence set forth in FIG. 1B or 1C, wherein the antibody comprises (i) a heavy chain modification that inhibits half antibody formation, (ii) a heavy chain modification that increases the antibody's terminal half-life, and (iii) a heavy chain modification that lowers the antibody's effector function.

This invention still further provides a humanized IgG2/IgG4 monoclonal fusion antibody having the light chain variable region amino acid sequence set forth in FIG. 1A and the heavy chain variable region amino acid sequence set forth in FIG. 1B or 1C, wherein (i) in the antibody, IgG2 (up to T260) is joined to IgG4 (with numbering according to the EU Index), and (ii) the antibody comprises a heavy chain modification that increases the antibody's terminal half-life.

This invention provides an isolated nucleic acid molecule encoding one or more chains of the present monoclonal antibody. This invention also provides a recombinant vector comprising the nucleotide sequence of the present nucleic acid molecule operably linked to a promoter of RNA transcription.

This invention also provides a composition comprising (i) the present monoclonal antibody, and (ii) a pharmaceutically acceptable carrier.

This invention further provides a recombinant AAV vector comprising a nucleic acid sequence encoding the heavy chain and/or the light chain of the present monoclonal antibody.

This invention still further provides a recombinant AAV particle comprising the present recombinant AAV vector and an AAV capsid protein, as well as a composition comprising (i) a plurality of the present AAV particles and (ii) a pharmaceutically acceptable carrier.

This invention provides a method for reducing the likelihood of a human subject's becoming infected with HIV-1 comprising administering to the subject a prophylactically effective amount of the present monoclonal antibody. This invention also provides a method for reducing the likelihood of a human subject's becoming infected with HIV-1 comprising administering to the subject a prophylactically effective number of the present recombinant AAV particles.

This invention provides a method for treating a human subject who is infected with HIV-1 comprising administering to the subject a therapeutically effective amount of the present monoclonal antibody. This invention also provides a method for treating a human subject who is infected with HIV-1 comprising administering to the subject a therapeutically effective number of the present recombinant AAV particles.

This invention provides a method for treating a human subject who is infected with SARS-CoV-2 comprising administering to the subject a therapeutically effective amount of the present monoclonal antibody. This invention also provides a method for treating a human subject who is infected with SARS-CoV-2 comprising administering to the subject a therapeutically effective number of the present recombinant AAV particles.

This invention provides a method for reducing the likelihood of a human subject's becoming afflicted with a CCR5-mediated disorder comprising administering to the subject a prophylactically effective amount of the present monoclonal antibody. This invention also provides a method for treating a human subject who is afflicted with a CCR5-mediated disorder comprising administering to the subject a therapeutically effective amount of the present monoclonal antibody. This invention further provides a method for reducing the likelihood of a human subject's becoming afflicted with a CCR5-mediated disorder comprising administering to the subject a prophylactically effective number of the present recombinant AAV particles. This invention still further provides a method for treating a human subject who is afflicted with a CCR5-mediated disorder comprising administering to the subject a therapeutically effective number of the present recombinant AAV particles. CCR5-mediated disorders include, without limitation, HIV-1 infection, hypercytokinemia, cytokine release syndrome, Alzheimer's disease, cancer (e.g., metastatic breast cancer), atherosclerosis, arthritis, inflammatory bowel disease, multiple sclerosis, and other immune-mediated illnesses such as graft-vs-host disease (GvHD) and non-alcoholic steatohepatitis (NASH).

This invention provides a first method for treating a human subject afflicted with a disorder comprising (i) administering to the subject a therapeutically effective amount of an agent for treating the disorder and (ii) administering to the subject a prophylactically effective amount of the present monoclonal antibody in conjunction with step (i), wherein the agent is known to cause cytokine release syndrome.

This invention also provides a second method for treating a human subject afflicted with a disorder comprising (i) administering to the subject a therapeutically effective amount of an agent for treating the disorder and (ii) administering to the subject a prophylactically effective number of the present AAV particles in conjunction with step (i), wherein the agent is known to cause cytokine release syndrome.

Finally, this invention provides three kits. The first kit comprises, in separate compartments, (a) a diluent and (b) a suspension of the present monoclonal antibody. The second kit comprises, in separate compartments, (a) a diluent and (b) the present monoclonal antibody in lyophilized form. The third kit comprises, in separate compartments, (a) a diluent and (b) a suspension of a plurality of the present recombinant AAV particles.

### BRIEF DESCRIPTION OF THE FIGURES

FIGS. 1A-1C

FIG. 1A sets forth the amino acid sequence of the light chain variable region of the antibodies “PRO 140 #1” and “PRO 140 #2” (as those antibodies are defined in U.S. Pat. No. 7,122,185 (the “'185 patent”)), as well as the nucleic acid sequence encoding it (corresponding to the sequences in FIG. 8 of the '185 patent). FIG. 1B sets forth the amino acid sequence of the heavy chain variable region of PRO 140 #2, as well as the nucleic acid sequence encoding it (corresponding to the sequences in FIG. 9 of the '185 patent). FIG. 1C sets forth the amino acid sequence of the heavy chain variable region of PRO 140 #1, as well as the nucleic acid sequence encoding it (corresponding to the sequences in FIG. 10 of the '185 patent).

FIG. 2

This figure shows a schematic diagram of an expression cassette encoding the present monoclonal antibody for inclusion in an AAV vector.

### DETAILED DESCRIPTION OF THE INVENTION

This invention provides certain recombinant monoclonal antibodies, recombinant viral vectors and particles, and related methods for addressing HIV-infection, SARS-CoV-2 infection, and other CCR5-mediated disorders like cancer.

### Definitions

In this application, certain terms are used which shall have the meanings set forth as follows.

As used herein, “administer”, with respect to monoclonal antibodies, means to deliver the antibodies to a subject's body via any known method suitable for that purpose. Specific modes of administration include, without limitation, intravenous, intramuscular, nasal, and subcutaneous administration. Preferably, the administration is subcutaneous. Similarly, as used herein, “administer”, with respect to recombinant viral particles, means to deliver the particles to a subject's body via any known method suitable for that purpose. Specific modes of administration include, without limitation, intravenous, intramuscular, and subcutaneous administration. Preferably, the administration is intravenous.

In this invention, monoclonal antibodies can be formulated using one or more routinely used pharmaceutically acceptable carriers. Such carriers are well known to those skilled in the art. For example, injectable drug delivery systems include solutions containing salts (e.g., sodium chloride and sodium phosphate). In a specific embodiment, the injectable drug delivery system comprises monoclonal antibody (e.g., 0.1 mg, 0.5 mg, 1 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,250 mg, 1,500 mg, 1,750 mg, 2,000 mg, 2,250 mg, 2,500 mg, 2,750 mg, 3,000 mg, 3,250 mg, 3,500 mg, 3,750 mg, 4,000 mg, 4,250 mg, 4,500 mg, 4,750 mg, 5,000 mg, 5,250 mg, 5,500 mg, 5,750 mg, 6,000 mg, 6,250 mg, 6,500 mg, 6,750 mg, 7,000 mg, 7,250 mg, 7,500 mg, 7,750 mg, 8,000 mg, 8,250 mg, 8,500 mg, 8,750 mg, 9,000 mg, 9,250 mg, 9,500 mg, 9,750 mg, or 10,000 mg) in the form of a lyophilized powder in a multi-use vial, which is then reconstituted and diluted in, for example, 0.9% Sodium Chloride Injection, USP. In another specific embodiment, the injectable drug delivery system comprises monoclonal antibody (e.g., 0.1 mg/50 ml, 0.5 mg/50 ml, 1 mg/50 ml, 5 mg/50 ml, 10 mg/50 ml, 25 mg/50 ml, 50 mg/50 ml, 75 mg/50 ml, 100 mg/50 ml, 125 mg/50 ml, 150 mg/50 ml, 175 mg/50 ml, 200 mg/50 ml, 225 mg/50 ml, 250 mg/50 ml, 275 mg/50 ml, 300 mg/50 ml, 325 mg/50 ml, 350 mg/50 ml, 375 mg/50 ml, 400 mg/50 ml, 425 mg/50 ml, 450 mg/50 ml, 475 mg/50 ml, 500 mg/50 ml, 525 mg/50 ml, 550 mg/50 ml, 575 mg/50 ml, 600 mg/50 ml, 625 mg/50 ml, 650 mg/50 ml, 675 mg/50 ml, 700 mg/50 ml, 725 mg/50 ml, 750 mg/50 ml, 775 mg/50 ml, 800 mg/50 ml, 825 mg/50 ml, 850 mg/50 ml, 875 mg/50 ml, 900 mg/50 ml, 925 mg/50 ml, 950 mg/50 ml, 975 mg/50 ml, 1,000 mg/50 ml, 1,250 mg/50 ml, 1,500 mg/50 ml, 1,750 mg/50 ml, 2,000 mg/50 ml, 2,250 mg/50 ml, 2,500 mg/50 ml, 2,750 mg/50 ml, 3,000 mg/50 ml, 3,250 mg/50 ml, 3,500 mg/50 ml, 3,750 mg/50 ml, 4,000 mg/50 ml, 4,250 mg/50 ml, 4,500 mg/50 ml, 4,750 mg/50 ml, 5,000 mg/50 ml, 5,250 mg/50 ml, 5,500 mg/50 ml, 5,750 mg/50 ml, 6,000 mg/50 ml, 6,250 mg/50 ml, 6,500 mg/50 ml, 6,750 mg/50 ml, 7,000 mg/50 ml, 7,250 mg/50 ml, 7,500 mg/50 ml, 7,750 mg/50 ml, 8,000 mg/50 ml, 8,250 mg/50 ml, 8,500 mg/50 ml, 8,750 mg/50 ml, 9,000 mg/50 ml, 9,250 mg/50 ml, 9,500 mg/50 ml, 9,750 mg/50 ml, or 10,000 mg/50 ml) in the form of a suspension in a single-use vial, which is then withdrawn and diluted in, for example, 0.9% Sodium Chloride Injection, USP. Injectable drug delivery systems also include suspensions, gels, microspheres and polymeric injectables, and can comprise excipients such as solubility-altering agents (e.g., ethanol, propylene glycol, and sucrose) and polymers (e.g., polycaprylactones and PLGAs).

In addition, in this invention, recombinant viral particles can be formulated using one or more routinely used pharmaceutically acceptable carriers. Such carriers are well known to those skilled in the art. For example, injectable drug delivery systems include solutions containing salts (e.g., sodium chloride and sodium phosphate) and surfactants (e.g., a poloxamer). In a specific embodiment, the injectable drug delivery system comprises an aqueous solution of sodium chloride (e.g., 180 mM), sodium phosphate (e.g., 10 mM), and a poloxamer (e.g., 0.001% Poloxamer 188). Injectable drug delivery systems also include suspensions, gels, microspheres and polymeric injectables, and can comprise excipients such as solubility-altering agents (e.g., ethanol, propylene glycol, and sucrose) and polymers (e.g., polycaprylactones and PLGAs).

As used herein, the term “antibody” includes, without limitation, (a) an immunoglobulin molecule comprising two heavy chains (i.e., H chains, such as μ, δ, γ, α and ε) and two light chains (i.e., L chains, such as A and K) and which recognizes an antigen; (b) polyclonal and monoclonal immunoglobulin molecules; (c) monovalent and divalent fragments thereof, and (d) bispecific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4 (preferably IgG2 and IgG4). Antibodies can be both naturally occurring and non-naturally occurring. Furthermore, antibodies include chimeric antibodies, wholly synthetic antibodies, single chain antibodies (e.g., scFv), and fragments thereof. Antibodies may contain, for example, all or a portion of a constant region (e.g., an Fc region) and a variable region, or contain only a variable region (responsible for antigen binding). Antibodies may be human, humanized, or nonhuman. Methods for designing and making humanized antibodies are well known (See, e.g., Chiu and Gilliland; Lafleur, et al.). Antibodies include, without limitation, the present monoclonal antibodies as defined herein.

“CCR5” (i.e., human CCR5), to which the present monoclonal antibody binds, is a member of the beta chemokine receptor family of integral membrane proteins. It is a G protein-coupled receptor (also known as a GPCR or seven-(pass)-transmembrane domain receptor) that functions as a chemokine receptor in the CC chemokine group. CCR5's ligands include CCL3 and CCL4 (also known as MIP 1α and 1β, respectively), CCL3L1, and CCR5 (also known as RANTES). These are small chemotactic cytokines that mediate chemo-attraction between cells.

As used herein, a “CCR5-mediated disorder” means a disorder wherein the presence of CCR5 in the afflicted subject plays a role in the disorder's formation, continuation, and/or progression. Examples of CCR5-mediated disorders include, without limitation, HIV-1 infection, hypercytokinemia, cytokine release syndrome, Alzheimer's disease, cancer (e.g., metastatic breast cancer), atherosclerosis, arthritis, inflammatory bowel disease, multiple sclerosis, and other immune-mediated illnesses such as graft-vs-host disease (GvHD) and non-alcoholic steatohepatitis (NASH). CCR5 plays a role in HIV-1 infection, in that it is a co-receptor for HIV-1 entry into, and infection of, target cells. PRO 140 binds tightly to CCR5 and is a powerful inhibitor of HIV-1 infection.

“Cytokine release syndrome” (CRS) is a potentially life-threatening systemic inflammatory response that follows the administration of certain therapeutic antibodies (e.g., Rituxan® (rituximab)) and adoptive T-cell therapies (e.g., Kymriah® (tisagenlecleucel)).

As used herein, “effector function”, with respect to an antibody, includes, without limitation, antibody-dependent cell-mediated cytotoxicity (ADCC), antibody-dependent cellular phagocytosis (ADCP), and complement fixation. Methods for measuring effector function are known (see, e.g., R. P. Rother, et al.; C. Dumet, et al.; T. Schlothauer, et al.; C.-H. Lee, et al.; and O. Vafa, et al.). These known methods include, by way of example, the (i) surface-plasmon-resonance analysis; (ii) solution-phase complement-activation assay; (iii) enzyme-linked immunosorbent assay; (iv) C1q cell surface binding assay; (v) complement-dependent cytotoxicity (CDC) assays; (vi) complementor FcγR-mediated cellular cytotoxicity assays; (vii) complement-receptor inhibition assays; (viii) phagocytosis assays (ADCP or CDCP); (ix) anaphylaxis assay; and (x) FcγRIIb-mediated internalization assay, all as described in C.-H. Lee, et al. These methods also include, for example, the methods for determining (i) ADCC and CDC; (ii) ADCP; and (iii) Fc-dependent induction of cytokine release (FIC), all as described in O. Vafa, et al.

As used herein, a subject who has been “exposed” to HIV-1 includes, for example, a subject who experienced a high-risk event (e.g., one in which he/she came into contact with the bodily fluids of an infected human subject). In one embodiment, this exposure occurs one month, three weeks, two weeks, one week, five days, four days, three days, two days or 24 hours prior to receiving the present prophylaxis.

As used herein, a “human subject” can be of any age, gender or state of co-morbidity. In one embodiment, the subject is male, and in another, the subject is female. In another embodiment, the subject is co-morbid (e.g., afflicted with diabetes, asthma, and/or heart disease). In a further embodiment, the subject is not co-morbid. In still another embodiment, the subject is younger than 60 years old. In yet another embodiment, the subject is at least 60 years old, at least 65 years old, at least 70 years old, at least 75 years old, at least 80 years old, at least 85 years old, or at least 90 years old.

“Hypercytokinemia”, also known as a cytokine storm, is a severe immune reaction in which the body releases too many cytokines into the blood too quickly. In an afflicted subject, large numbers of white blood cells are activated and release inflammatory cytokines, which in turn activate yet more white blood cells. Hypercytokinemia can occur, for example, as a result of viral infection, as is the case with SARS-CoV-2, Ebola, and MERS-CoV. In SARS-CoV-2-infected subjects, pulmonary involvement leads to acute respiratory distress syndrome (ARDS), a life-threatening illness. ARDS results in shock, multi-organ failure including cardiac failure, and eventually death. For a subject infected with SARS-CoV-2, CCR5 blockade by the present monoclonal antibody can both inhibit the onset of, and reduce the severity of, hypercytokinemia.

As used herein, a heavy chain modification that “increases” the terminal half-life of the present monoclonal antibody is a modification that renders the present monoclonal antibody's terminal half-life longer than that of PRO 140 when administered at the same dose (e.g., 700 mg) and by the same route (e.g., subcutaneously, intravenously, or intramuscularly). In a preferred embodiment, the present monoclonal antibody's terminal half-life is longer than that of PRO 140 (when administered at the same dose and by the same route) by at least a factor of two, at least a factor of three, at least a factor of four, at least a factor of five, at least a factor of six, at least a factor of seven, at least a factor of eight, at least a factor of nine, at least a factor of 10, at least a factor of 15, at least a factor of 20, at least a factor of 25, at least a factor of 30, at least a factor of 35, at least a factor of 40, at least a factor of 45, or at least a factor of 50. In another preferred embodiment, the present monoclonal antibody's terminal half-life is at least two weeks, at least one month, at least two months, or at least three months. In a further preferred embodiment, the present monoclonal antibody's terminal half-life is at least five days, at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 90 days, at least 95 days, or at least 100 days. In a further preferred embodiment, the present monoclonal antibody's terminal half-life is from five days to 10 days, from 10 days to 15 days, from 15 days to 20 days, from 20 days to 25 days, from 25 days to 30 days, from 30 days to 35 days, from 35 days to 40 days, from 40 days to 45 days, from 45 days to 50 days, from 50 days to 55 days, from 55 days to 60 days, from 60 days to 65 days, from 65 days to 70 days, from 70 days to 75 days, from 75 days to 80 days, from 80 days to 85 days, from 85 days to 90 days, from 90 days to 95 days, from 95 days to 100 days, or over 100 days. An antibody's terminal half-life (e.g., its mean terminal half-life) can be determined, for example, based on data from in vivo human studies, and can also be determined, for example, based on data from animal studies (e.g., studies in mice (e.g., human FcRn transgenic mice available from Jackson Labs (see, e.g., G. Proetzel and D. C. Roopenian; Avery, et al.; and D. Sheridan, et al.)), rats, rabbits, and monkeys (such as rhesus monkeys, cynamolgous macaques, and marmosets)). Methods for determining an antibody's terminal half-life are known (see, e.g., P. L. Toutain and A. Bousquet-Melou). Examples of heavy chain modifications that increase antibody terminal half-life (such as those that increase antibody binding to FcRn) are described in C. Dumet, et al. and G. J. Robbie, et al. They include, without limitation, the following, with numbering according to the EU Index: (i) point mutations at position 252, 254, 256, 309, 311, 433, 434, and/or 436, including the “YTE” mutation combination M252Y/S254T/T256E (U.S. Pat. No. 7,083,784); (ii) the “LS” mutation combination M428L/N434S (WO/2009/086320); (iii) the “QL” mutation combination T250Q/M428L; and (iv) the mutation combinations M428L/V308F and Q311V/N434S. By way of example, the heavy chain modification M252Y/S254T/T256E (YTE) increases the terminal half-life of the present monoclonal antibody if the present monoclonal antibody's terminal half-life is 10 days and that of PRO 140 is three days, when the present monoclonal antibody and PRO 140 are both administered subcutaneously at a 700 mg dose.

As used herein, a subject is “infected” with a virus if the virus is present in the subject. Present in the subject includes, without limitation, present in at least some cells in the subject, and/or present in at least some extracellular fluid in the subject. In one embodiment, the virus present in the subject's cells is replicating. A subject who is exposed to a virus may or may not become infected with it.

Heavy chain modifications that “inhibit half antibody formation” in IgG4 are described, for example, in C. Dumet, et al. They include, without limitation, the following, with numbering according to the EU Index: (i) S228P; (ii) the mutation combination S228P/R409K, and (iii) the mutation combination S228P/K447del. Moreover, the mutations 447del and 446/447del are known to reduce IgG4 C-terminal heterogeneity, as described in C. Dumet.

As used herein, a heavy chain modification that “lowers the effector function” of the present monoclonal antibody is a modification that renders the present monoclonal antibody's effector function lower than that of PRO 140. These effector function-lowering heavy chain modifications include, without limitation, IgG4 heavy chain point mutations and deletion mutations. Examples of IgG4 heavy chain modifications that lower effector function relative to wild-type IgG4 heavy chains are described in C. Dumet, et al. They include, without limitation, the following, with numbering according to the EU Index: (i) L235E (WO/1994/028027); (ii) L235A, F234A, and G237A (WO/1994/029351 and WO/1995/026403); (iii) D265A (U.S. Pat. No. 7,332,581); (iv) L328 substitution, A330R, and F243L (WO/2004/029207); (v) IgG2/IgG4 format wherein IgG2 (up to T260) is joined to IgG4 (WO/2005/007809); (vi) F243A/V264A combination (WO/2011/149999); (vii) E233P/F234A/L235A/G236del/G237A combination (WO/2017/079369); and (viii) S228P/L235E combination.

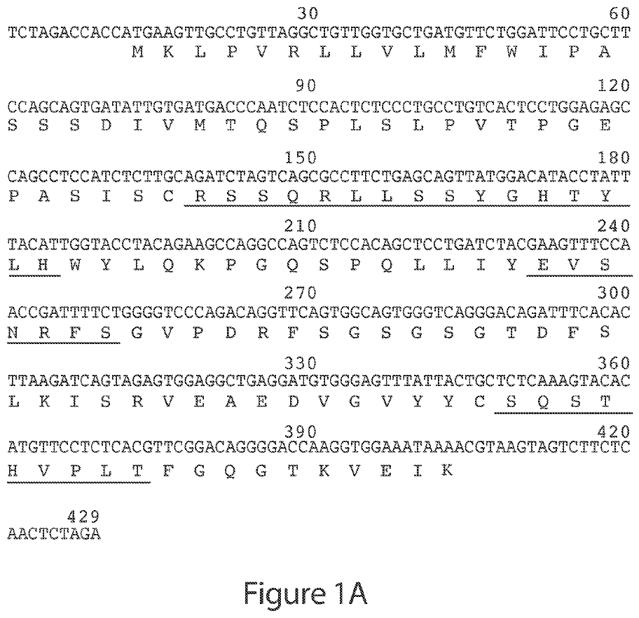

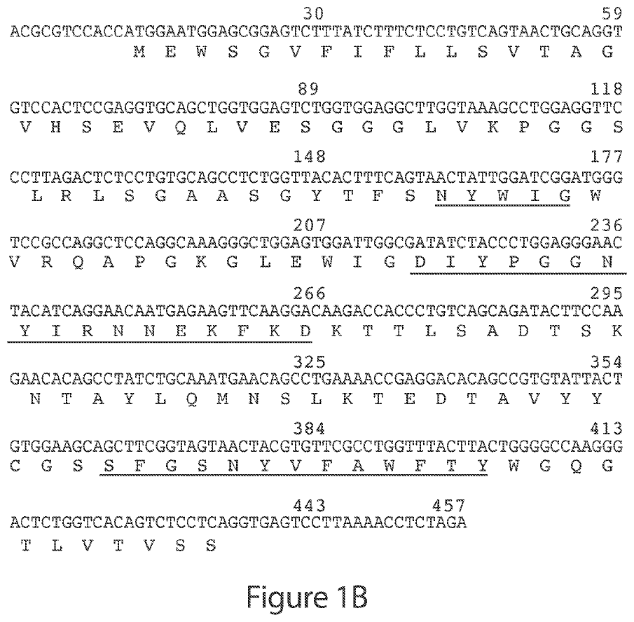

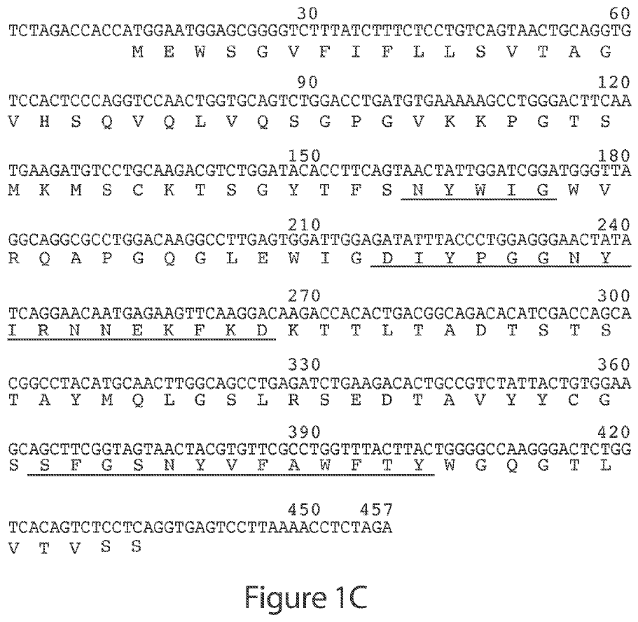

“PRO 140 #1” and “PRO 140 #2”, as defined in U.S. Pat. No. 7,122,185, are referred to herein, collectively and individually, as “PRO 140.” PRO 140 #1 has the heavy and light chain variable region amino acid sequences set forth in FIGS. 1A and 1C (which correspond, respectively, to the heavy chain variable region amino acid sequence encoded by pVg4:HuPRO140 (mut B+D+I)-VH (ATCC Deposit Designation PTA-4099), and to the light chain variable region amino acid sequence encoded by pVK:HuPRO140-VK (ATCC Deposit Designation PTA-4097)). PRO 140 #2 has the heavy and light chain variable region amino acid sequences set forth in FIGS. 1A and 1B (which correspond, respectively, to the heavy chain variable region amino acid sequence encoded by pVg4:HuPRO140 HG2-VH (ATCC Deposit Designation PTA-4098), and to the light chain variable region amino acid sequence encoded by pVK:HuPRO140-VK (ATCC Deposit Designation PTA-4097)). Without limiting the present invention, it is noted that PRO 140 binds to human CCR5 expressed, for example, on T helper cells, monocytes, macrophages and dendritic cells. PRO 140 was designed to potently inhibit HIV-1 entry in the majority of new infections (i.e., macrophage-tropic HIV-1 or CCR5-tropic HIV-1 exclusively).

As used herein, a “prophylactically effective amount” of the present monoclonal antibodies includes, without limitation, (i) 0.1 mg, 0.5 mg, 1 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,250 mg, 1,500 mg, 1,750 mg, 2,000 mg, 2,250 mg, 2,500 mg, 2,750 mg, 3,000 mg, 3,250 mg, 3,500 mg, 3,750 mg, 4,000 mg, 4,250 mg, 4,500 mg, 4,750 mg, 5,000 mg, 5,250 mg, 5,500 mg, 5,750 mg, 6,000 mg, 6,250 mg, 6,500 mg, 6,750 mg, 7,000 mg, 7,250 mg, 7,500 mg, 7,750 mg, 8,000 mg, 8,250 mg, 8,500 mg, 8,750 mg, 9,000 mg, 9,250 mg, 9,500 mg, 9,750 mg, or 10,000 mg; (ii) 0.1 mg to 1 mg, 1 mg to 5 mg, 5 mg to 20 mg, 20 mg to 50 mg, 50 mg to 100 mg, 100 mg to 150 mg, 150 mg to 200 mg, 200 mg to 250 mg, 250 mg to 300 mg, 300 mg to 350 mg, 350 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1,000 mg, 1,000 mg to 1,250 mg, 1,250 mg to 1,500 mg, 1,500 mg to 1,750 mg, 1,750 mg to 2,000 mg, 2,000 mg to 2,250 mg, 2,250 mg to 2,500 mg, 2,500 mg to 2,750 mg, 2,750 mg to 3,000 mg, 3,000 mg to 3,250 mg, 3,250 mg to 3,500 mg, 3,500 mg to 3,750 mg, 3,750 mg to 4,000 mg, 4,000 mg to 4,250 mg, 4,250 mg to 4,500 mg, 4,500 mg to 4,750 mg, 4,750 mg to 5,000 mg, 5,000 mg to 5,250 mg, 5,250 mg to 5,500 mg, 5,500 mg to 5,750 mg, 5,750 mg to 6,000 mg, 6,000 mg to 6,250 mg, 6,250 mg to 6,500 mg, 6,500 mg to 6,750 mg, 6,750 mg to 7,000 mg, 7,000 mg to 7,250 mg, 7,250 mg to 7,500 mg, 7,500 mg to 7,750 mg, 7,750 mg to 8,000 mg, 8,000 mg to 8,250 mg, 8,250 mg to 8,500 mg, 8,500 mg to 8,750 mg, 8,750 mg to 9,000 mg, 9,000 mg to 9,250 mg, 9,250 mg to 9,500 mg, 9,500 mg to 9,750 mg, or 9,750 mg to 10,000 mg; (iii) 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 200 mg/kg, 300 mg/kg, 400 mg/kg, or 500 mg/kg; or (iv) 0.1 mg/kg to 1 mg/kg, 1 mg/kg to 10 mg/kg, 10 mg/kg to 20 mg/kg, 20 mg/kg to 30 mg/kg, 30 mg/kg to 40 mg/kg, 40 mg/kg to 50 mg/kg, 50 mg/kg to 60 mg/kg, 60 mg/kg to 70 mg/kg, 70 mg/kg to 80 mg/kg, 80 mg/kg to 90 mg/kg, 90 mg/kg to 100 mg/kg, 100 mg/kg to 200 mg/kg, 200 mg/kg to 300 mg/kg, 300 mg/kg to 400 mg/kg, or 400 mg/kg to 500 mg/kg. In the preferred embodiment, the prophylactically effective amount of monoclonal antibodies is 175 mg, 350 mg, or 700 mg, administered subcutaneously. In another preferred embodiment, the prophylactically effective amount of monoclonal antibodies is administered as a single, one-time-only dose. In a further embodiment, the prophylactically effective amount of monoclonal antibodies is administered as two or more doses over a period of days, weeks, or months (e.g., twice daily for one or two weeks; once daily for one or two weeks; every other day for two weeks; three times per week for two weeks; twice per week for two weeks; once per week for two weeks; twice with the administrations separated by two weeks; once per month; once every two months; once every three months; once every four months; twice per year; or once per year).

In a preferred embodiment, the prophylactically effective amount of the present monoclonal antibodies is administered according to a regimen for pre-exposure prophylaxis (“PrEP”) for the prevention of HIV infection. PrEP involves the regular (e.g., daily, weekly, monthly, semiannual, or annual) use of anti-retroviral therapy before and after events involving the risk of HIV exposure, in order to lower the chances of acquiring HIV infection. These events include sex and intravenous drug use, for example. The CDC provides a clinical practice guideline for PrEP (“Preexposure Prophylaxis for the Prevention of HIV Infection in the United States—2021 Update”), and PrEP is also the subject of ongoing research (see, e.g., J. Riddell IV, et al.; H. Yusuf, et al.; and R. Chou, et al.). In addition, preferred embodiments of PrEP for the present monoclonal antibodies are set forth in the Examples section below.

As used herein, a “prophylactically effective amount” of the present recombinant viral particles (e.g., recombinant AAV particles) includes, without limitation, (i) from 1×10¹⁰ to 5×10¹⁰ particles (also referred to as “viral genomes” or “vg”) per kg of body weight, from 5×10¹⁰ to 1×10¹¹ particles/kg, from 1×10¹¹ to 5×10¹¹ particles/kg, from 5×10¹¹ to 1×10¹² particles/kg, from 1×10¹² to 5×10¹² particles/kg, from 5×10¹² to 1×10¹³ particles/kg, from 1×10¹³ to 5×10¹³ particles/kg, or from 5×10¹³ to 1×10¹⁴ particles/kg; or (ii) 1×10¹⁰ particles/kg, 5×10¹⁰ particles/kg, 1×10¹¹ particles/kg, 5×10¹¹ particles/kg, 1×10¹² particles/kg, 5×10¹² particles/kg, 1×10¹³ particles/kg, 5×10¹³ particles/kg, or 1×10¹⁴ particles/kg, 5×10¹⁴ particles/kg, or 1×10¹⁵ particles/kg. In the preferred embodiment, the prophylactically effective amount of viral particles is administered as a single, one-time-only dose. In another embodiment, the prophylactically effective amount of viral particles is administered as two or more doses over a period of months or years.

As used herein, a “recombinant AAV (adeno-associated virus) particle”, also referred to as “rAAV particle”, includes, without limitation, an AAV capsid protein (e.g., VP1, VP2 and/or VP3) and a vector comprising a nucleic acid encoding an exogenous protein (e.g., an antibody heavy chain) situated between a pair of AAV inverted terminal repeats in a manner permitting the AAV particle to infect a target cell. Preferably, the recombinant AAV particle is incapable of replication within its target cell. The AAV serotype may be any AAV serotype suitable for use in gene therapy, such as AAV1, AAV2, AAV3, AAV4, AAV5, AAV6, AAV7, AAV8, AAV9, AAV10, AAVrh10, AAV11, AAV12, LK01, LK02 or LK03.

As used herein, “reducing the likelihood” of a human subject's becoming infected with a virus includes, without limitation, reducing such likelihood by at least 10%, at least 20%, at least 30%, at least 40%, at least 50%, at least 60%, at least 70%, at least 80%, at least 90%, at least 95%, or at least 99%. Preferably, reducing the likelihood of a human subject's becoming infected with a virus means preventing the subject from becoming infected with it. Similarly, “reducing the likelihood” of a human subject's becoming symptomatic of a viral infection includes, without limitation, reducing such likelihood by at least 10%, at least 20%, at least 30%, at least 40%, at least 50%, at least 60%, at least 70%, at least 80%, at least 90%, at least 95%, or at least 99%. Preferably, reducing the likelihood of a human subject's becoming symptomatic of a viral infection means preventing the subject from becoming symptomatic.

As used herein, the term “subject” includes, without limitation, a mammal such as a human, a non-human primate, a dog, a cat, a horse, a sheep, a goat, a cow, a rabbit, a pig, a hamster, a rat and a mouse. The present methods are envisioned for these non-human embodiments, mutatis mutandis, as they are for human subjects in this invention.

As used herein, a human subject is “symptomatic” of an HIV-1 infection if the subject shows one or more symptoms known to appear in an HIV-1-infected human subject after a suitable incubation period. Such symptoms include, without limitation, detectable HIV-1 in the subject, and those symptoms shown by patients afflicted with AIDS. AIDS-related symptoms include, without limitation, weight loss, fever, fatigue and opportunistic infections.

As used herein, a human subject is “symptomatic” of a SARS-CoV-2 infection if the subject shows one or more symptoms known to appear in a SARS-CoV-2-infected human subject after a suitable incubation period. Such symptoms include, without limitation, detectable SARS-CoV-2 in the subject, and those symptoms shown by patients afflicted with COVID-19. COVID-19-related symptoms include, without limitation, fever, cough, shortness of breath, persistent pain or pressure in the chest, new confusion or inability to arouse, and/or bluish lips or face.

As used herein, a “therapeutically effective amount” of the present monoclonal antibodies includes, without limitation, (i) 0.1 mg, 0.5 mg, 1 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,250 mg, 1,500 mg, 1,750 mg, 2,000 mg, 2,250 mg, 2,500 mg, 2,750 mg, 3,000 mg, 3,250 mg, 3,500 mg, 3,750 mg, 4,000 mg, 4,250 mg, 4,500 mg, 4,750 mg, 5,000 mg, 5,250 mg, 5,500 mg, 5,750 mg, 6,000 mg, 6,250 mg, 6,500 mg, 6,750 mg, 7,000 mg, 7,250 mg, 7,500 mg, 7,750 mg, 8,000 mg, 8,250 mg, 8,500 mg, 8,750 mg, 9,000 mg, 9,250 mg, 9,500 mg, 9,750 mg, or 10,000 mg; (ii) 0.1 mg to 1 mg, 1 mg to 5 mg, 5 mg to 20 mg, 20 mg to 50 mg, 50 mg to 100 mg, 100 mg to 150 mg, 150 mg to 200 mg, 200 mg to 250 mg, 250 mg to 300 mg, 300 mg to 350 mg, 350 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1,000 mg, 1,000 mg to 1,250 mg, 1,250 mg to 1,500 mg, 1,500 mg to 1,750 mg, 1,750 mg to 2,000 mg, 2,000 mg to 2,250 mg, 2,250 mg to 2,500 mg, 2,500 mg to 2,750 mg, 2,750 mg to 3,000 mg, 3,000 mg to 3,250 mg, 3,250 mg to 3,500 mg, 3,500 mg to 3,750 mg, 3,750 mg to 4,000 mg, 4,000 mg to 4,250 mg, 4,250 mg to 4,500 mg, 4,500 mg to 4,750 mg, 4,750 mg to 5,000 mg, 5,000 mg to 5,250 mg, 5,250 mg to 5,500 mg, 5,500 mg to 5,750 mg, 5,750 mg to 6,000 mg, 6,000 mg to 6,250 mg, 6,250 mg to 6,500 mg, 6,500 mg to 6,750 mg, 6,750 mg to 7,000 mg, 7,000 mg to 7,250 mg, 7,250 mg to 7,500 mg, 7,500 mg to 7,750 mg, 7,750 mg to 8,000 mg, 8,000 mg to 8,250 mg, 8,250 mg to 8,500 mg, 8,500 mg to 8,750 mg, 8,750 mg to 9,000 mg, 9,000 mg to 9,250 mg, 9,250 mg to 9,500 mg, 9,500 mg to 9,750 mg, or 9,750 mg to 10,000 mg; (iii) 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 200 mg/kg, 300 mg/kg, 400 mg/kg, or 500 mg/kg; or (iv) 0.1 mg/kg to 1 mg/kg, 1 mg/kg to 10 mg/kg, 10 mg/kg to 20 mg/kg, 20 mg/kg to 30 mg/kg, 30 mg/kg to 40 mg/kg, 40 mg/kg to 50 mg/kg, 50 mg/kg to 60 mg/kg, 60 mg/kg to 70 mg/kg, 70 mg/kg to 80 mg/kg, 80 mg/kg to 90 mg/kg, 90 mg/kg to 100 mg/kg, 100 mg/kg to 200 mg/kg, 200 mg/kg to 300 mg/kg, 300 mg/kg to 400 mg/kg, or 400 mg/kg to 500 mg/kg. In the preferred embodiment, the therapeutically effective amount of monoclonal antibodies is 175 mg, 350 mg, or 700 mg, administered subcutaneously. In another preferred embodiment, the therapeutically effective amount of monoclonal antibodies is administered as a single, one-time-only dose. In a further embodiment, the therapeutically effective amount of monoclonal antibodies is administered as two or more doses over a period of days, weeks, or months (e.g., twice daily for one or two weeks; once daily for one or two weeks; every other day for two weeks; three times per week for two weeks; twice per week for two weeks; once per week for two weeks; twice with the administrations separated by two weeks; once per month; once every two months; once every three months; once every four months; twice per year; or once per year).

As used herein, a “therapeutically effective amount” of the present recombinant viral particles (e.g., recombinant AAV particles) includes, without limitation, (i) from 1×10¹⁰ to 5×10¹⁰ particles (also referred to as “viral genomes” or “vg”) per kg of body weight, from 5×10¹⁰ to 1×10¹¹ particles/kg, from 1×10¹¹ to 5×10¹¹ particles/kg, from 5×10¹¹ to 1×10¹² particles/kg, from 1×10¹² to 5×10¹² particles/kg, from 5×10¹² to 1×10¹³ particles/kg, from 1×10¹³ to 5×10¹³ particles/kg, or from 5×10¹³ to 1×10¹⁴ particles/kg; or (ii) 1×10¹⁰ particles/kg, 5×10¹⁰ particles/kg, 1×10¹¹ particles/kg, 5×10¹¹ particles/kg, 1×10¹² particles/kg, 5×10¹² particles/kg, 1×10¹³ particles/kg, 5×10¹³ particles/kg, or 1×10¹⁴ particles/kg, 5×10¹⁴ particles/kg, or 1×10¹⁵ particles/kg. In the preferred embodiment, the therapeutically effective amount of viral particles is administered as a single, one-time-only dose. In another embodiment, the therapeutically effective amount of viral particles is administered as two or more doses over a period of months or years.

As used herein, “treating” a subject afflicted with a disorder (e.g., infected with SARS-CoV-2 and symptomatic of that infection; infected with HIV-1 and symptomatic of that infection; or afflicted with another CCR5-mediated disorder) includes, without limitation, (i) slowing, stopping, or reversing the progression of one or more of the disorder's symptoms, (ii) slowing, stopping or reversing the progression of the disorder underlying such symptoms, (iii) reducing or eliminating the likelihood of the symptoms' recurrence, and/or (iv) slowing the progression of, lowering or eliminating the disorder. In the preferred embodiment, treating a subject afflicted with a disorder includes (i) reversing the progression of one or more of the disorder's symptoms, (ii) reversing the progression of the disorder underlying such symptoms, (iii) preventing the symptoms' recurrence, and/or (iv) eliminating the disorder. For a subject infected with SARS-CoV-2 but not symptomatic of that infection, “treating” the subject also includes, without limitation, reducing the likelihood of the subject's becoming symptomatic of the infection, and preferably, preventing the subject from becoming symptomatic of the infection. When the disorder is cancer, treating a subject includes, without limitation, slowing, stopping, or reversing metastasis.

### EMBODIMENTS OF THE INVENTION

This invention provides certain anti-CCR5 monoclonal antibodies. These antibodies possess the light and heavy chain variable region amino acid sequences of PRO 140 (leronlimab), and differ from PRO 140 with respect to certain heavy chain modifications that inhibit half antibody formation, increase terminal half-life, and lower the effector function. It also provides recombinant viral particles (preferably recombinant AAV particles) that, when introduced into a subject, cause the long-term expression of those antibodies. These antibodies and viral particles address SARS-CoV-2 infection, HIV-1 infection, and CCR5-related diseases.

Specifically, this invention provides a humanized monoclonal antibody having the light chain variable region amino acid sequence set forth in FIG. 1A and the heavy chain variable region amino acid sequence set forth in FIG. 1B or 1C, wherein the antibody comprises a heavy chain modification that increases the antibody's terminal half-life. In one embodiment, this monoclonal antibody is an immunoglobulin molecule comprising two heavy chains (e.g., μ, δ, γ, α, or ε) and two light chains (e.g., λ or κ), and derives from IgA, secretory IgA, IgM, and IgG subclasses (e.g., human IgG1, IgG2, IgG3, or IgG4 (preferably IgG2 or IgG4)).

This invention also provides a first humanized IgG4 monoclonal antibody having the light chain variable region amino acid sequence set forth in FIG. 1A and the heavy chain variable region amino acid sequence set forth in FIG. 1B or 1C, wherein the antibody comprises (i) a heavy chain modification that inhibits half antibody formation, and (ii) a heavy chain modification that increases the antibody's terminal half-life. The first humanized IgG4 monoclonal antibody is preferably a humanized IgG4κ monoclonal antibody, but may also be a humanized IgG4λ monoclonal antibody.

In one embodiment, the first humanized IgG4 (preferably IgG4κ) monoclonal antibody has the light chain variable region amino acid sequence set forth in FIG. 1A and the heavy chain variable region amino acid sequence set forth in FIG. 1B. In another embodiment, the first humanized IgG4 (preferably IgG4κ) monoclonal antibody has the light chain variable region amino acid sequence set forth in FIG. 1A and the heavy chain variable region amino acid sequence set forth in FIG. 1C.

In a preferred embodiment of the first humanized IgG4 (preferably IgG4κ) monoclonal antibody, the half antibody formation-inhibiting mutation is selected from the group consisting of S228P, the S228P/R409K combination, and the S228P/K447del combination (with numbering according to the EU Index).

In another preferred embodiment of the first humanized IgG4 (preferably IgG4κ) monoclonal antibody, the antibody comprises a terminal half-life-extending mutation combination selected from the group consisting of M252Y/S254T/T256E (YTE) and M428L/N434S (LS) (with numbering according to the EU Index).

This invention provides a humanized monoclonal antibody having the light chain variable region amino acid sequence set forth in FIG. 1A and the heavy chain variable region amino acid sequence set forth in FIG. 1B or 1C, wherein the antibody comprises (i) a heavy chain modification that increases the antibody's terminal half-life, and (ii) a heavy chain modification that lowers the antibody's effector function. In one embodiment, this monoclonal antibody is an immunoglobulin molecule comprising two heavy chains (e.g., μ, δ, γ, α, or ε) and two light chains (e.g., λ or κ), and derives from IgA, secretory IgA, IgM, and IgG subclasses (e.g., human IgG1, IgG2, IgG3, or IgG4 (preferably IgG2 or IgG4)).

This invention also provides a second humanized IgG4 monoclonal antibody having the light chain variable region amino acid sequence set forth in FIG. 1A and the heavy chain variable region amino acid sequence set forth in FIG. 1B or 1C, wherein the antibody comprises (i) a heavy chain modification that inhibits half antibody formation, (ii) a heavy chain modification that increases the antibody's terminal half-life, and (iii) a heavy chain modification that lowers the antibody's effector function. The second humanized IgG4 monoclonal antibody is preferably a humanized IgG4κ monoclonal antibody, but may also be a humanized IgG4λ monoclonal antibody.

In one embodiment, the second humanized IgG4 (preferably IgG4κ) monoclonal antibody has the light chain variable region amino acid sequence set forth in FIG. 1A and the heavy chain variable region amino acid sequence set forth in FIG. 1B. In another embodiment, the second humanized IgG4 (preferably IgG4κ) monoclonal antibody has the light chain variable region amino acid sequence set forth in FIG. 1A and the heavy chain variable region amino acid sequence set forth in FIG. 1C.

In a preferred embodiment of the second humanized IgG4 (preferably IgG4κ) monoclonal antibody, the half antibody formation-inhibiting mutation is selected from the group consisting of S228P, the S228P/R409K combination, and the S228P/K447del combination (with numbering according to the EU Index).

In another preferred embodiment of the second humanized IgG4 (preferably IgG4κ) monoclonal antibody, the antibody comprises a terminal half-life-extending mutation combination selected from the group consisting of M252Y/S254T/T256E (YTE) and M428L/N434S (LS) (with numbering according to the EU Index).

In a further preferred embodiment of the second humanized IgG4 (preferably IgG4κ) monoclonal antibody, the antibody comprises an effector function-lowering mutation selected from the group consisting of L235E, L235A, F234A, G237A, D265A, an L328 substitution, A330R, F243L, the F243A/V264A combination, the E233P/F234A/L235A/G236del/G237A combination, and the S228P/L235E combination (with numbering according to the EU Index).

This invention further provides a humanized IgG2/IgG4 monoclonal fusion antibody having the light chain variable region amino acid sequence set forth in FIG. 1A and the heavy chain variable region amino acid sequence set forth in FIG. 1B or 1C, wherein (i) in the antibody, IgG2 (up to T260) is joined to IgG4 (with numbering according to the EU Index), and (ii) the antibody comprises a heavy chain modification that increases the antibody's terminal half-life. The humanized IgG2/IgG4 monoclonal fusion antibody is preferably a humanized IgG2/IgG4κ monoclonal fusion antibody, but may also be a humanized IgG2/IgG4λ monoclonal fusion antibody.

In one embodiment, the humanized IgG2/IgG4 (preferably IgG2/IgG4κ) monoclonal fusion antibody has the light chain variable region amino acid sequence set forth in FIG. 1A and the heavy chain variable region amino acid sequence set forth in FIG. 1B. In another embodiment, the humanized IgG2/IgG4 (preferably IgG2/IgG4κ) monoclonal fusion antibody has the light chain variable region amino acid sequence set forth in FIG. 1A and the heavy chain variable region amino acid sequence set forth in FIG. 1C.

In a preferred embodiment of the humanized IgG2/IgG4 (preferably IgG2/IgG4κ) monoclonal fusion antibody, the antibody comprises a terminal half-life-extending mutation combination selected from the group consisting of M252Y/S254T/T256E (YTE) and M428L/N434S (LS) (with numbering according to the EU Index).

The above monoclonal antibodies are referred to herein, collectively and individually, as the present monoclonal antibody.

The following exemplary antibodies are envisioned as embodiments of the present monoclonal antibody. The first exemplary antibody is a humanized IgG4 (preferably IgG4κ) monoclonal antibody that (i) has the terminal half-life-extending mutation combination M252Y/S254T/T256E (YTE); and (ii) has the half antibody formation-inhibiting mutation S228P (all with numbering according to the EU Index). In one embodiment, the first exemplary antibody has the effector function-lowering L235E mutation (with numbering according to the EU Index). In another embodiment, the first exemplary antibody has one or more of the effector function-lowering mutations L235A, F234A, and G237A (with numbering according to the EU Index). In a further embodiment, the first exemplary antibody has the effector function-lowering D265A mutation (with numbering according to the EU Index). In a further embodiment, the first exemplary antibody has one or more of the effector function-lowering mutations A330R, F243L, and an L328 substitution (with numbering according to the EU Index). In a further embodiment, the first exemplary antibody has the effector function-lowering F243A/V264A mutation combination (with numbering according to the EU Index). In a further embodiment, the first exemplary antibody has the effector function-lowering E233P/F234A/L235A/G236del/G237A mutation combination (with numbering according to the EU Index). In yet a further embodiment, the first exemplary antibody has the effector function-lowering S228P/L235E mutation combination (with numbering according to the EU Index).

The second exemplary antibody is a humanized IgG4 (preferably IgG4κ) monoclonal antibody that (i) has the terminal half-life-extending mutation combination M252Y/S254T/T256E (YTE); and (ii) has the half antibody formation-inhibiting mutation combination S228P/R409K (all with numbering according to the EU Index). In one embodiment, the second exemplary antibody has the effector function-lowering L235E mutation (with numbering according to the EU Index). In another embodiment, the second exemplary antibody has one or more of the effector function-lowering mutations L235A, F234A, and G237A (with numbering according to the EU Index). In a further embodiment, the second exemplary antibody has the effector function-lowering D265A mutation (with numbering according to the EU Index). In a further embodiment, the second exemplary antibody has one or more of the effector function-lowering mutations A330R, F243L, and an L328 substitution (with numbering according to the EU Index). In a further embodiment, the second exemplary antibody has the effector function-lowering F243A/V264A mutation combination (with numbering according to the EU Index). In a further embodiment, the second exemplary antibody has the effector function-lowering E233P/F234A/L235A/G236del/G237A mutation combination (with numbering according to the EU Index). In yet a further embodiment, the second exemplary antibody has the effector function-lowering S228P/L235E mutation combination (with numbering according to the EU Index).

The third exemplary antibody is a humanized IgG4 (preferably IgG4κ) monoclonal antibody that (i) has the terminal half-life-extending mutation combination M252Y/S254T/T256E (YTE); and (ii) has the half antibody formation-inhibiting mutation combination S228P/K447del (all with numbering according to the EU Index). In one embodiment, the third exemplary antibody has the effector function-lowering L235E mutation (with numbering according to the EU Index). In another embodiment, the third exemplary antibody has one or more of the effector function-lowering mutations L235A, F234A, and G237A (with numbering according to the EU Index). In a further embodiment, the third exemplary antibody has the effector function-lowering D265A mutation (with numbering according to the EU Index). In a further embodiment, the third exemplary antibody has one or more of the effector function-lowering mutations A330R, F243L, and an L328 substitution (with numbering according to the EU Index). In a further embodiment, the third exemplary antibody has the effector function-lowering F243A/V264A mutation combination (with numbering according to the EU Index). In a further embodiment, the third exemplary antibody has the effector function-lowering E233P/F234A/L235A/G236del/G237A mutation combination (with numbering according to the EU Index). In yet a further embodiment, the third exemplary antibody has the effector function-lowering S228P/L235E mutation combination (with numbering according to the EU Index).

The fourth exemplary antibody is a humanized IgG4 (preferably IgG4κ) monoclonal antibody that (i) has the terminal half-life-extending mutation combination M428L/N434S (LS); and (ii) has the half antibody formation-inhibiting mutation S228P (all with numbering according to the EU Index). In one embodiment, the fourth exemplary antibody has the effector function-lowering L235E mutation (with numbering according to the EU Index). In another embodiment, the fourth exemplary antibody has one or more of the effector function-lowering mutations L235A, F234A, and G237A (with numbering according to the EU Index). In a further embodiment, the fourth exemplary antibody has the effector function-lowering D265A mutation (with numbering according to the EU Index). In a further embodiment, the fourth exemplary antibody has one or more of the effector function-lowering mutations A330R, F243L, and an L328 substitution (with numbering according to the EU Index). In a further embodiment, the fourth exemplary antibody has the effector function-lowering F243A/V264A mutation combination (with numbering according to the EU Index). In a further embodiment, the fourth exemplary antibody has the effector function-lowering E233P/F234A/L235A/G236del/G237A mutation combination (with numbering according to the EU Index). In yet a further embodiment, the fourth exemplary antibody has the effector function-lowering S228P/L235E mutation combination (with numbering according to the EU Index).

The fifth exemplary antibody is a humanized IgG4 (preferably IgG4κ) monoclonal antibody that (i) has the terminal half-life-extending mutation combination M428L/N434S (LS); and (ii) has the half antibody formation-inhibiting mutation combination S228P/R409K (all with numbering according to the EU Index). In one embodiment, the fifth exemplary antibody has the effector function-lowering L235E mutation (with numbering according to the EU Index). In another embodiment, the fifth exemplary antibody has one or more of the effector function-lowering mutations L235A, F234A, and G237A (with numbering according to the EU Index). In a further embodiment, the fifth exemplary antibody has the effector function-lowering D265A mutation (with numbering according to the EU Index). In a further embodiment, the fifth exemplary antibody has one or more of the effector function-lowering mutations A330R, F243L, and an L328 substitution (with numbering according to the EU Index). In a further embodiment, the fifth exemplary antibody has the effector function-lowering F243A/V264A mutation combination (with numbering according to the EU Index). In a further embodiment, the fifth exemplary antibody has the effector function-lowering E233P/F234A/L235A/G236del/G237A mutation combination (with numbering according to the EU Index). In yet a further embodiment, the fifth exemplary antibody has the effector function-lowering S228P/L235E mutation combination (with numbering according to the EU Index).

The sixth exemplary antibody is a humanized IgG4 (preferably IgG4κ) monoclonal antibody that (i) has the terminal half-life-extending mutation combination M428L/N434S (LS); and (ii) has the half antibody formation-inhibiting mutation combination S228P/K447del (all with numbering according to the EU Index). In one embodiment, the sixth exemplary antibody has the effector function-lowering L235E mutation (with numbering according to the EU Index). In another embodiment, the sixth exemplary antibody has one or more of the effector function-lowering mutations L235A, F234A, and G237A (with numbering according to the EU Index). In a further embodiment, the sixth exemplary antibody has the effector function-lowering D265A mutation (with numbering according to the EU Index). In a further embodiment, the sixth exemplary antibody has one or more of the effector function-lowering mutations A330R, F243L, and an L328 substitution (with numbering according to the EU Index). In a further embodiment, the sixth exemplary antibody has the effector function-lowering F243A/V264A mutation combination (with numbering according to the EU Index). In a further embodiment, the sixth exemplary antibody has the effector function-lowering E233P/F234A/L235A/G236del/G237A mutation corn bination (with numbering according to the EU Index). In yet a further embodiment, the sixth exemplary antibody has the effector function-lowering S228P/L235E mutation combination (with numbering according to the EU Index).

The seventh exemplary antibody is a humanized IgG2/IgG4 (preferably IgG2/IgG4κ) monoclonal fusion antibody that has the terminal half-life-extending mutation combination M428L/N434S (LS) (with numbering according to the EU Index).

The eighth exemplary antibody is a humanized IgG2/IgG4 (preferably IgG2/IgG4κ) monoclonal fusion antibody that has the terminal half-life-extending mutation combination M252Y/S254T/T256E (YTE) (with numbering according to the EU Index).

This invention provides an isolated nucleic acid molecule encoding one or more chains of the present monoclonal antibody. In one embodiment, the present nucleic acid molecule is a DNA molecule, for example, a cDNA molecule.

This invention further provides a recombinant vector, for example a plasmid or a viral vector, comprising the present nucleic acid molecule operably linked to a promoter of RNA transcription.

This invention still further provides a host vector system comprising one or more of the present vectors in a suitable host cell (e.g., a bacterial cell, an insect cell, a yeast cell, or a mammalian cell such as a hybridoma cell (See, e.g., Chiu and Gilliland; Kohler and Milstein)). This invention further provides a method for producing the present monoclonal antibody comprising culturing the present host vector system.

In connection with the present vectors, a nucleic acid sequence “encoding” a protein (e.g., an antibody heavy chain) encodes it operably (i.e., in a manner permitting its expression in a cell infected by a viral particle comprising the vector that contains the nucleic acid sequence). Additionally, the recombinant viral vectors of this invention are not limited to any particular configuration with respect to the exogenous protein-coding sequences. For example, in one embodiment of the present recombinant AAV vector, a “one vector” approach is used wherein a singular recombinant AAV vector includes nucleic acid sequences encoding both heavy and light antibody chains. In another embodiment, a “two vector” approach is used wherein one recombinant AAV vector includes a nucleic acid sequence encoding the heavy antibody chain, and a second recombinant AAV vector includes a nucleic acid sequence encoding the light antibody chain (See, e.g., S. P. Fuchs, et al.).

This invention provides a composition comprising (i) the present monoclonal antibody, and (ii) a pharmaceutically acceptable carrier.

This invention also provides a recombinant AAV vector comprising a nucleic acid sequence encoding the heavy chain and/or the light chain of the present monoclonal antibody.

In a preferred embodiment of the present recombinant AAV vector, the nucleic acid sequence encodes the heavy chain and the light chain of the present monoclonal antibody.

This invention further provides a recombinant AAV particle comprising the present recombinant AAV vector and an AAV capsid protein.

This invention still further provides a composition comprising (i) a plurality of the present AAV particles and (ii) a pharmaceutically acceptable carrier.

This invention provides a method for reducing the likelihood of a human subject's becoming infected with HIV-1 comprising administering to the subject a prophylactically effective amount of the present monoclonal antibody. Preferably, this method comprises administering the present monoclonal antibody according to a PrEP regimen. This invention also provides a method for reducing the likelihood of a human subject's becoming infected with HIV-1 comprising administering to the subject a prophylactically effective number of the present recombinant AAV particles. In the preferred embodiment of each of these prophylactic methods, the subject has been exposed to HIV-1.

This invention provides a method for treating a human subject who is infected with HIV-1 comprising administering to the subject a therapeutically effective amount of the present monoclonal antibody. This invention also provides a method for treating a human subject who is infected with HIV-1 comprising administering to the subject a therapeutically effective number of the present recombinant AAV particles. In the preferred embodiment of each of these therapeutic methods, the subject is symptomatic of an HIV-1 infection.

This invention provides a method for treating a human subject who is infected with SARS-CoV-2 comprising administering to the subject a therapeutically effective amount of the present monoclonal antibody. This invention also provides a method for treating a human subject who is infected with SARS-CoV-2 comprising administering to the subject a therapeutically effective number of the present recombinant AAV particles. In one embodiment of each of these therapeutic methods, the subject is symptomatic of a SARS-CoV-2 infection. In another embodiment, the subject is asymptomatic of a SARS-CoV-2 infection.

In addition to the prophylactic and therapeutic methods above, this invention provides prophylactic and therapeutic methods for addressing CCR5-mediated disorders generally. Specifically, this invention provides a method for reducing the likelihood of a human subject's becoming afflicted with a CCR5-mediated disorder comprising administering to the subject a prophylactically effective amount of the present monoclonal antibody. This invention also provides a method for treating a human subject who is afflicted with a CCR5-mediated disorder comprising administering to the subject a therapeutically effective amount of the present monoclonal antibody. This invention further provides a method for reducing the likelihood of a human subject's becoming afflicted with a CCR5-mediated disorder comprising administering to the subject a prophylactically effective number of the present recombinant AAV particles. This invention still further provides a method for treating a human subject who is afflicted with a CCR5-mediated disorder comprising administering to the subject a therapeutically effective number of the present recombinant AAV particles. In one embodiment of each of these prophylactic and therapeutic methods, the CCR5-mediated disorder is selected from the group consisting of hypercytokinemia, cytokine release syndrome, Alzheimer's disease, cancer, atherosclerosis, arthritis, inflammatory bowel disease, non-alcoholic steatohepatitis (NASH), graft-vs-host disease (GvHD), and multiple sclerosis. Preferably, the CCR5-mediated disorder is metastatic breast cancer.

The following are four exemplary embodiments of the present methods for addressing CCR5-mediated disorders. In a first embodiment, this invention provides a method for reducing the likelihood of a human subject's becoming afflicted with hypercytokinemia comprising administering to the subject a prophylactically effective amount of the present monoclonal antibody. Preferably, the subject is infected with a virus (e.g., SARS-CoV-2).

In a second embodiment, this invention provides a method for treating a human subject who is afflicted with hypercytokinemia comprising administering to the subject a therapeutically effective amount of the present monoclonal antibody. Preferably, the subject is infected with a virus (e.g., SARS-CoV-2).

In a third embodiment, this invention provides a method for reducing the likelihood of a human subject's becoming afflicted with hypercytokinemia comprising administering to the subject a prophylactically effective number of the present recombinant AAV particles. Preferably, the subject is infected with a virus (e.g., SARS-CoV-2).

In a fourth embodiment, this invention provides a method for treating a human subject who is afflicted with hypercytokinemia comprising administering to the subject a therapeutically effective number of the present recombinant AAV particles. Preferably, the subject is infected with a virus (e.g., SARS-CoV-2).

This invention provides methods for treating a human subject afflicted with a disorder (such as cancer) whereby the present monoclonal antibody or AAV particle is administered to the subject in conjunction with an agent for treating the disorder, wherein the agent is known to cause cytokine release syndrome. This either prevents the agent from causing cytokine release syndrome in the subject, or reduces the severity of any cytokine release syndrome that the agent causes.

Specifically, this invention provides a first method for treating a human subject afflicted with a disorder (such as cancer) comprising (i) administering to the subject a therapeutically effective amount of an agent for treating the disorder (e.g., a monoclonal antibody or an adoptive T-cell therapy) and (ii) administering to the subject a prophylactically effective amount of the present monoclonal antibody in conjunction with step (i), wherein the agent is known to cause cytokine release syndrome.

In a preferred embodiment of the first method, the disorder is selected from the group consisting of non-Hodgkin's lymphoma (NHL), chronic lymphocytic leukemia (CLL), and rheumatoid arthritis, and the agent is Rituxan® (rituximab). In another preferred embodiment, the disorder is selected from the group consisting of B-cell precursor acute lymphoblastic leukemia, diffuse large B-cell lymphoma (DLBCL) not otherwise specified, high grade B-cell lymphoma, and DLBCL arising from follicular lymphoma, and the agent is Kymriah® (tisagenlecleucel).

In the first method, administering the present monoclonal antibody to the subject in conjunction with the agent includes, for example, (i) administering the present monoclonal antibody to the subject before administering the agent, (ii) administering the present monoclonal antibody to the subject concurrently with the agent (e.g., as an admixture or via separate infusions), or (iii) administering the present monoclonal antibody to the subject immediately after administering the agent. In one embodiment, administering the present monoclonal antibody to the subject in conjunction with the agent includes (i) and (ii) above; (ii) and (iii) above; (i) and (iii) above; or (i), (ii), and (iii) above. Preferably, the first method prevents cytokine release syndrome in the subject, or at least reduces its severity.

This invention also provides a second method for treating a human subject afflicted with a disorder (such as cancer) comprising (i) administering to the subject a therapeutically effective amount of an agent for treating the disorder (e.g., a monoclonal antibody or an adoptive T-cell therapy) and (ii) administering to the subject a prophylactically effective number of the present AAV particles in conjunction with step (i), wherein the agent is known to cause cytokine release syndrome.

In a preferred embodiment of the second method, the disorder is selected from the group consisting of non-Hodgkin's lymphoma (NHL), chronic lymphocytic leukemia (CLL), and rheumatoid arthritis, and the agent is Rituxan® (rituximab). In another preferred embodiment, the disorder is selected from the group consisting of B-cell precursor acute lymphoblastic leukemia, diffuse large B-cell lymphoma (DLBCL) not otherwise specified, high grade B-cell lymphoma, and DLBCL arising from follicular lymphoma, and the agent is Kymriah® (tisagenlecleucel).

In the second method, administering the present AAV particles to the subject in conjunction with the agent includes, for example, administering the present AAV particles to the subject before administering the agent. Preferably, the second method prevents cytokine release syndrome in the subject, or at least reduces its severity.

Finally, this invention provides three kits. The first kit comprises, in separate compartments, (a) a diluent and (b) a suspension of the present monoclonal antibody. The second kit comprising, in separate compartments, (a) a diluent and (b) the present monoclonal antibody in lyophilized form. The third kit comprises, in separate compartments, (a) a diluent and (b) a suspension of a plurality of the present recombinant AAV particles. In one example, the third kit comprises (i) a single-dose vial containing a concentrated solution of the present particles (also measured as viral genomes) in a suitable solution (e.g., a solution of sterile water, sodium chloride, sodium phosphate, and Poloxamer 188) and (ii) one or more vials of suitable diluent (e.g., a solution of sterile water, sodium chloride, sodium phosphate, and Poloxamer 188).

The present vectors, particles, and methods are envisioned for suitable recombinant non-AVV viruses (e.g., lentivirus, adenovirus, alphavirus, herpesvirus, or vaccinia virus), mutatis mutandis, as they are for recombinant AAV viruses in this invention.

The present compositions, vectors, viral particles, methods, and kits are envisioned for the IgM and IgA antibody classes of the present monoclonal antibodies, mutatis mutandis, as they are for the IgG antibody class in this invention.

This invention will be better understood by reference to the examples which follow, but those skilled in the art will readily appreciate that the specific examples detailed are only illustrative of the invention as described more fully in the claims which follow thereafter.

### EXAMPLES

### Example 1—Determining Terminal Half-Life

In a preferred embodiment, the terminal half-life of the present monoclonal antibody can be determined using the homozygous human FcRn transgenic mouse referred to as “Tg32” and described in Avery, et al. Specifically, a single dose (e.g., 10 mg/kg) of the present monoclonal antibody is intravenously administered to the Tg32 mouse, and the antibody's terminal half-life in the mouse is determined. Preferably, a single dose (e.g., 10 mg/kg) of the present monoclonal antibody and a single dose of the same amount of PRO 140 are separately (e.g., concurrently) intravenously administered to Tg32 mice, so that the terminal half-life of each can be determined and compared. Methods for determining terminal half-life based on in vivo data are known and are described, for example, in D. Sheridan, et al. By way of example, plasma terminal half-life can be determined using Pharsight Phoenix WinNonlin software.

### Example 2—PrEP Regimens

In a preferred embodiment of this invention, the present monoclonal antibodies are prophylactically administered according to a PrEP regimen in order to more effectively lower the likelihood of a subject's becoming infected with HIV during a high-risk event such as sex or intravenous drug use.

Subjects

In one embodiment, a subject who would benefit from a PrEP regimen using the present monoclonal antibodies includes a sexually active individual who has engaged in anal or vaginal sex within six months of initiating a PrEP regimen, and (i) has an HIV-positive sexual partner, and/or (ii) has a history inconsistent condom use, or no condom use, with sexual partners.

Dosages

A suitable amount of the present monoclonal antibody is administered as a single 3 ml subcutaneous injection. In one embodiment, the initial dose is followed by a second dose after two weeks, and by further doses every two weeks thereafter, with follow-up visits as appropriate. In another embodiment, the initial dose is followed by a second dose after one month, and by further doses every two months thereafter, with follow-up visits as appropriate. In another embodiment, the initial dose is followed by a second dose after two months, and by further doses every three months thereafter, with follow-up visits as appropriate. In a further embodiment, the initial dose is followed by a second dose after three months, and by further doses every three months thereafter, with follow-up visits as appropriate. In yet a further embodiment, the initial dose is followed by a second dose after six months, and by further doses every six months thereafter, with follow-up visits as appropriate.

The suitable amount of the present monoclonal antibody includes, without limitation, any of the following: (i) 0.1 mg, 0.5 mg, 1 mg, 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,250 mg, 1,500 mg, 1,750 mg, 2,000 mg, 2,250 mg, 2,500 mg, 2,750 mg, 3,000 mg, 3,250 mg, 3,500 mg, 3,750 mg, 4,000 mg, 4,250 mg, 4,500 mg, 4,750 mg, 5,000 mg, 5,250 mg, 5,500 mg, 5,750 mg, 6,000 mg, 6,250 mg, 6,500 mg, 6,750 mg, 7,000 mg, 7,250 mg, 7,500 mg, 7,750 mg, 8,000 mg, 8,250 mg, 8,500 mg, 8,750 mg, 9,000 mg, 9,250 mg, 9,500 mg, 9,750 mg, or 10,000 mg; (ii) 0.1 mg to 1 mg, 1 mg to 5 mg, 5 mg to 20 mg, 20 mg to 50 mg, 50 mg to 100 mg, 100 mg to 150 mg, 150 mg to 200 mg, 200 mg to 250 mg, 250 mg to 300 mg, 300 mg to 350 mg, 350 mg to 400 mg, 400 mg to 450 mg, 450 mg to 500 mg, 500 mg to 550 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1,000 mg, 1,000 mg to 1,250 mg, 1,250 mg to 1,500 mg, 1,500 mg to 1,750 mg, 1,750 mg to 2,000 mg, 2,000 mg to 2,250 mg, 2,250 mg to 2,500 mg, 2,500 mg to 2,750 mg, 2,750 mg to 3,000 mg, 3,000 mg to 3,250 mg, 3,250 mg to 3,500 mg, 3,500 mg to 3,750 mg, 3,750 mg to 4,000 mg, 4,000 mg to 4,250 mg, 4,250 mg to 4,500 mg, 4,500 mg to 4,750 mg, 4,750 mg to 5,000 mg, 5,000 mg to 5,250 mg, 5,250 mg to 5,500 mg, 5,500 mg to 5,750 mg, 5,750 mg to 6,000 mg, 6,000 mg to 6,250 mg, 6,250 mg to 6,500 mg, 6,500 mg to 6,750 mg, 6,750 mg to 7,000 mg, 7,000 mg to 7,250 mg, 7,250 mg to 7,500 mg, 7,500 mg to 7,750 mg, 7,750 mg to 8,000 mg, 8,000 mg to 8,250 mg, 8,250 mg to 8,500 mg, 8,500 mg to 8,750 mg, 8,750 mg to 9,000 mg, 9,000 mg to 9,250 mg, 9,250 mg to 9,500 mg, 9,500 mg to 9,750 mg, or 9,750 mg to 10,000 mg; (iii) 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 200 mg/kg, 300 mg/kg, 400 mg/kg, or 500 mg/kg; or (iv) 0.1 mg/kg to 1 mg/kg, 1 mg/kg to 10 mg/kg, 10 mg/kg to 20 mg/kg, 20 mg/kg to 30 mg/kg, 30 mg/kg to 40 mg/kg, 40 mg/kg to 50 mg/kg, 50 mg/kg to 60 mg/kg, 60 mg/kg to 70 mg/kg, 70 mg/kg to 80 mg/kg, 80 mg/kg to 90 mg/kg, 90 mg/kg to 100 mg/kg, 100 mg/kg to 200 mg/kg, 200 mg/kg to 300 mg/kg, 300 mg/kg to 400 mg/kg, or 400 mg/kg to 500 mg/kg.

### Example 3—Inhibition of Mucosal SHIV Transmission in Macaques

In a preferred embodiment of this invention, the prophylactic efficacy of the present monoclonal antibodies is tested in macaques using the following method taken from Chang, et al.

HIV and SHIV Stocks

HIV-1 isolates are obtained from the NIH AIDS Reagent Program, with the majority from the HIV-1 60 International Isolate Panel (Cat #11412). The SHIV_{SF162P3} stock (173.3 ng/mL p27, 1×10⁹ vRNA copies/mL, 2.67×10⁵ TCID₅₀/mL in TZM-bl cells, 1.28×10³ TCID₅₀/mL in rhesus PBMC (peripheral blood mononuclear cells)) is used in the PrEP animal study.

HIV and SI-11V In Vitro Infection Assays

Human PBMCs are first depleted of CD8+ T cells with Human CD8 Microbeads (Miltenyi) and then sequentially enriched for CD4+ T cells with Human CD4 Microbeads (Miltenyi) following the manufacturer's instructions. The resulting CD4+CD8− T cells are incubated at 2×10⁶ cells/mL in R15-100 media (RPMI 1640 with antibiotic/mycotic, 15% fetal bovine serum (FBS), and 100 U/mL IL-2) and activated for 24 hours with a stimulating cocktail containing CD3, CD49d, CD28 antibodies (BD Biosciences), and Staphylococcal enterotoxin B (Toxin Technologies, Inc.). After 24 hours, cells are washed two times and incubated for 2-3 additional days in R15-100 before viral infection. At day 0 of infection, 5×10⁵ cells are incubated with or without the desired concentration of mAb (i.e., the present monoclonal antibody) for 1 hour at 37° C. Next, cells are infected with the desired HIV isolate by spinoculation for 2 hours at 1200×g at room temperature (RT). Cells are washed four times with R15-100 to remove free viruses and cultured with R15-100 media plus the same concentration of mAb used during the pre-treatment step. An additional 5×10⁵ cells are left uninfected and kept in culture as the uninfected control. Cultures are maintained by replacing 50% of the culture with new media containing the desired concentration of mAb every other day for 5 days, when cells are harvested for intracellular p24 staining by flow cytometry.

Rhesus macaque (RM) PBMCs are depleted of CD8+ T cells by staining with NHP CD8-PE (Miltenyi) followed by anti-PE microbeads (Miltenyi) then subsequently enriched for CD4+ T cells with NHP CD4 microbeads (Miltenyi), following the manufacturer's instructions. Purified RM CD4+ cells are activated and maintained in culture similarly to human CD4+ T cells, as described above. For the spreading assay with HIV-1 Ba-L, HIV-1 LAI, and SHIV_{SF162P3}, CD4+ T cells are isolated, activated, infected, and cultured as described above but with the following changes. HIV-1 Ba-L, HIV-1 LAI, and SHIVsn62p3 are used to infect at a multiplicity of infection (MOI) of 1×10⁻⁵ (FFU/cell) and kept in culture for 6 days before p24 or p27 intracellular staining. For detection of intracellular HIV p24 or SHIV p27 by flow cytometry, cells are stained for CD3, CD4, CD8, and amine-reactive dye for viability for 30 minutes at RT in the dark. Cells are washed once with phosphate-buffered saline (PBS), spun down at 830×g for 4 minutes, and fixed with 2% paraformaldehyde (PFA) for 30 minutes in 4° C. Afterwards, cells are washed once with FACS buffer (10% bovine growth serum in PBS) and stained with p24 or p27 antibodies in 100 μL of Permeabilization Medium B (Thermo Fisher) for 1 hour at RT in the dark. Cells are washed once with FACS buffer and fixed again with 2% PFA for more than 30 minutes before collecting on LSR-II instrument and FACsDIVA version 6.1 (BD Biosciences, Franklin Lakes, N.J.). Data are analyzed using FlowJo v10 (Tree Star) by gating on singlet, live, CD3+, CD8−, and p24+ or p27+.

Human Blood Donors

Healthy human donor whole blood is purchased from Innovative Research and further processed to PBMC by density gradient centrifugation using Ficoll-Hypague. Blood is collected with K2 EDTA anticoagulant and tested negative for the following viral markers: HIV-1 RNA, antibodies to HIV, antibodies to hepatitis C virus (HCV), HCV RNA, hepatitis B virus (HBV) DNA, hepatitis B surface antigen (HbsAg), and syphilis. CCR5 expression is confirmed via flow cytometry. Leukapheresis samples are collected from TRB following the relevant informed consent procedures.

Quantitation of CCR5 Expression Levels

To measure the frequency of CCR5-expressing cells, PBMCs are incubated with 5 μg/mL of unlabeled mAb for 30 minutes at RT in the dark, and then washed once with PBS. Anti-human IgG4 is used as a secondary antibody to detect surface-bound mAb for 30 minutes at RT in the dark. Cells are washed once with FACS buffer and once with PBS, and then stained for CD3, CD4, CD8, CCR5 (via antibody clone 3A9, which is specific to a distinct, non-competitive CCR5 epitope than mAb), and amine-reactive dye for 30 minutes at RT in the dark. Cells are washed twice with PBS and fixed with 2% PFA before collecting through the LSR-II instrument and FACsDIVA version 6.1. Samples are analyzed by gating on singlet, live, CD3+, CD4+/CD8−, and CCR5+(via clone 3A9) and/or human IgG4+ events. The number of CCR5 molecules on the cell surface is measured with quantitative cytometry using the Quantum Molecules of Equivalent Soluble Fluorochrome (MESF) kit (Bangs Laboratories, Inc). PE-conjugated mAb used to quantify surface CCR5 expression and PE-labeled microspheres for standard curve generation are provided by IncelIDX. Phenotypic staining is done using CD3−, CD4−, CD8−, CD14−, CD16− specific antibodies used as described above. T cell memory subset determination is defined as central memory (human: CCR7+CD45RA−, RM: CD28+CD95+), effector memory (human: CCR7−CD45RA−, RM: CD28−CD95+), and naive (human: CCR7+CD45RA+, RM: CD28+CD95−). Gating is done using FlowJo v10. MFI is used to attain MESF according to the manufacturer's protocol.

mAb CCR5 RO

To measure the percentage of CCR5 RO on the surface of CD4+ T cells, the following RO equation can be employed:

% RO=[% IgG4/(% IgG4+% mAb-PB)]×100%.

The equation measures unoccupied CCR5 receptors by using Pacific Blue-conjugated mAb (termed mAb-PB). CCR5 RO is defined as the percentage of cells CCR5+(measured by clone 3A9) and mAb+(measured by anti-human IgG4) divided by the percentage of cells CCR5+ and mAb+(measured by the sum of anti-human IgG4 and mAb-PB) cells following incubation with a saturating concentration of mAb-PB. This method is based on RO assays for anti-PD-1 antibodies in clinical trials.

PBMC or single cells from tissue homogenates (0.3-1×10⁶) are stained with anti-human IgG4 for 30 minutes at RT in the dark. Next, cells are washed once with FACS buffer and three times with PBS and then stained with CD45, CD3, CD4, CD8, CD16, CD14, amine-reactive dye, CCR5 (via antibody clone 3A9), and mAb-PB for 30 minutes at RT in the dark. Finally, cells are washed twice with PBS and fixed with 2% PFA for more than 30 minutes before collecting on LSR-II instrument and FACsDIVA version 6.1. Using FlowJo v10, cells are gated on CD45+, singlet, live, CD3+, CD4+, and CCR5+(via 3A9 staining) events. The CD4+ CCR5+ population is further gated on human IgG4+ or mAb-PB+ events.

Rhesus Macaques

All study RMs are housed in ABSL-2+ rooms with autonomously controlled temperature, humidity, and lighting. At assignment, all study RM are free of cercopithicine herpesvirus 1, D-type simian retrovirus, simian T-lymphotrophic virus type 1, and Mycobacterium tuberculosis. RMs are typed for the MHC alleles Mamu-Aantibody01, Mamu-Aantibody02, Mamu-Bantibodyl7, and Mamu-Bantibody08, with Mamu-Bantibodyl7 and/or -Bantibody08 positive animals excluded when possible or placed into control groups when not possible to exclude biasing results. All attempts are made to pair housed RM during the study period. When RMs are single cage-housed due to infection status, they had visual, auditory, and olfactory contact with other animals, and an enhanced enrichment plan is designed and overseen by RM behavior specialists. RMs are fed commercially prepared primate chow twice daily and receive supplemental fresh fruit or vegetables daily. Fresh, potable water is provided via automatic water systems. RMs are sedated with ketamine HCl or dexmedetomidine for procedures, including subcutaneous mAb administration, venipuncture, BAL, tissue biopsy, and SHIV challenge. For spleen and mesenteric lymph node biopsies, animals are sedated with isoflurane. At scheduled endpoints, RMs are euthanized with sodium pentobarbital overdose (>50 mg/kg) and exsanguinated via the distal aorta. A certified veterinary pathologist performs tissue collection at necropsy. The relevant authorities should approve RM care and all experimental protocols and procedures.

For the mAb PrEP study, a total of 18 adult RMs are used and divided between three experimental groups: (1) six RMs in the control group, (2) six RMs in the 10 mg/kg weekly mAb-treated group, and (3) six RMs in the 50 mg/kg every 2 weeks mAb-treated group. The three groups are gender matched, with two females and four male RMs in each group. Animals receive their first injection of mAb 1 week before the initiation of viral challenges, and continue to receive mAb until study week 7, for a total of nine mAb injections for group 1 (10 mg/kg treated group) and a total of five mAb injections for group 2 (50 mg/kg treated group). At study week 0, all three groups receive their first IR SHIV_{SF162P3} challenge with a 1:400 dilution delivered via atraumatic installation into the rectum with a needleless syringe. Viral challenge then continues every week until confirmed infection or until study week seven for a total of eight consecutive weekly IR challenges. Animals are euthanized as described above, with the timing dictated by the following guidelines: (1) 10 weeks after acquisition of SHIV_{SF162P3} infection, which is confirmed by plasma viremia, or (2) 8 weeks after complete washout of plasma mAb and loss of mAb CCR5 RO on CD4+ T cells in blood. Three SHIV-naïve RMs serve as adoptive transfer recipients for tissue homogenates from PrEP RM as described. All mAb used in these studies is clinical-grade material provided at a concentration of 175 mg/mL.

Processing of Blood and Tissue

Whole blood is collected into EDTA-treated or non-anticoagulant tubes (BD Biosciences). Blood in EDTA-tubes is assessed for complete blood counts using an ABX Pentra 60C+ Hematology Analyzer. Blood in non-anticoagulant tubes is spun down at 1860×g for 10 minutes to separate serum from clotted blood, and then assessed for serum chemistry values using an ABX Pentra 400 Chemistry Analyzer. PBMCs and plasma are isolated from whole blood in EDTA-treated tubes by density gradient centrifugation using Ficoll-Hypaque. Bronchoalveolar lavage fluid is collected in PBS and filtered through 70-μm cell strainers. Lymph node and spleen are collected in RPMI 1640 containing 10% FBS (R10), diced into tiny pieces with a scalpel, and forced through 70-μm cell strainers to collect single-cell suspensions. Bone marrow is collected in R10, pelleted by spinning at 830×g for 4 minutes. Cell pellet is resuspended by vigorously shaking in PBS containing 2 nM EDTA. Cell suspension is spun down again at 830×g for 4 minutes and resuspended in 70% isotonic Percoll (GE Healthcare, Buckinghamshire, UK) before underlaying in 37% isotonic Percoll. Cells are spun at 500×g for 20 minutes with brake. Mononuclear cells in the center interface are collected and washed in R10. Duodenum and colon are collected in R10 and diced into tiny pieces with a scalpel. Tissues are placed into a 50 mL conical tube containing R3 (RPMI 1640 and 3% FBS) with 0.5 M EDTA, and then incubated in 37° C. with 225 r.p.m. shaking for 30 minutes to remove mucus coating. After 30 minutes, cells are poured over a tea strainer and sequentially washed three times with Hank's Buffered Salt Solution (HBSS) to remove EDTA and mucus coating. Tissues are returned into a 50 mL conical tube containing R3, 0.2 mg/mL collagenase (Sigma-Aldrich, St. Louis, Mo.), and 0.2 mg/mL DNase I (Roche, Indianapolis, Ind.), and then incubated in 37° C. with 225 r.p.m. shaking for 1 h. Digested tissues are filtered through a 70-μm cell strainers. Cell flow-throughs are spun at 840×g for 4 minutes. Pellet is resuspended in 70% isotonic Percoll and underlaid in 37% isotonic Percoll, before spinning at 500×g for 20 minutes with brake. Mononuclear cells in the center interface are collected and washed in R10.

To quantify mAb concentration and ADA in the blood, plasma is heat-inactivated by incubation at 56° C. for 30 minutes, and then spun for 20 minutes at 12,000×g to pellet residual debris. Resulting supernatant is transferred to a new tube and stored at −80° C. until assayed. To quantify mAb concentration in tissues, tissues are finely diced and 10-1,000 mg of tissue of interest are placed into Lysing Matrix tubes (MP Biomedicals) and 300-500 μL of complete EDTA-free Protease Inhibitor Cocktail (Sigma-Aldrich) in PBS is added. Tissue disruption is achieved by beating in a Beadbeater (Biospec) device for three cycles of 1 minute beating and 1 minute on ice. Supernatant from the tissue homogenate is transferred to a new tube and spun for 20 minutes at 12,000×g to pellet residual debris. Resulting supernatant is transferred to a new tube and stored at −80° C. until assayed.

Viral Nucleic Acid Detection

Nucleic acids from plasma and PBMC cell pellets are extracted using the Maxwell 16 instrument (Promega, Madison, Wis.) following the manufacturer's protocol, which uses the LEV Viral Nucleic Acid Kit for plasma and the LEV Whole Blood Nucleic Acid kit for cell pellets.

Nucleic acid from tissues is extracted by first placing tissues in Lysing Matrix tubes (MP Biomedicals) with 1 mL Tri-reagent (Molecular Research Center, TR-118). Then, it is vortexed to soaked tissues in Tri-reagent and placed on wet ice. Tissues are grinded using the MagNA Lyser rotor (Roche Life Science) for 1-2 cycles depending on the size of the tissue, alternating between the MagNA Lyser rotor and wet ice. Tissue homogenates are briefly spun down to pellet the beads and supernatant is pipetted out for nucleic acid extraction. Supernatant is mixed with 1/10 bromochloropropane (BCP) to Tri-reagent volume. Mixture is vortexed and incubated for 5 minutes in room temperature, before spinning at 12,000×g for 15 minutes at 4° C. to achieve phase separation. For RNA extraction, the upper aqueous layer is pipetted into a new tube containing 12 μL of glycogen (Thermo Scientific) and 0.5 mL isopropanol. The sample is inverted to mix and spun at 15,000×g for 10 minutes in room temperature. Isopropanol is carefully discarded without losing pellet, and then it is washed with 0.7 mL 75% ethanol twice by spinning at 15,000×g for 10 minutes at room temperature. After discarding the ethanol, the RNA pellet is dried at room temperature for 10-15 minutes. Finally, RNA resuspension buffer (10 mM Tris, pH 8) is added to the RNA-containing tube and incubated in a 37° C. heat block for 15 minutes to elute. Resuspended RNA is stored at −80° C. until assayed. For DNA extraction, the interphase and organic layers, not used for RNA extraction, are mixed with 0.5 mL of DNA extraction buffer, containing 4 M guanidine thiocyanate (Sigma), 50 mM sodium citrate (Sigma), and 1 M Tris base. The reaction is vortexed and incubated for 5 minutes in room temperature, before spinning at 12,000×g for 15 minutes at 4° C. to achieve phase separation. DNA-containing upper aqueous layer is pipetted into a new tube containing 12 μL of glycogen (Thermo Scientific) and 0.4 mL isopropanol. The remaining steps follow RNA extraction protocol described above, with the exception of the DNA resuspension buffer (10 mM Tris, pH 9) used to elute the DNA pellet.

Viral copies are measured by quantitative reverse transcription PCR (RT-qPCR) or qPCR that targets a highly conserved sequence of Gag. The assays use the SGAG21 forward primer (GTCTGCGTCATPTGGTGCATTC), SGAG22 reverse primer (CACTAGKTGTCTCTGCACTATPTGTTTTG), and pSGAG23 probe (5′-6-carboxyfluorescein [FAM]-CTTCPTCAGTKTGTTTCACTTTCTCTTCTGCG-black hole quencher [BHQ1]-3′). All viral detection assays are performed by those who are blinded regarding the treatment conditions of each animal.

To quantitate SHIV viral RNA in plasma, RT-qPCR reactions are performed using the TaqMan Fast Virus 1-Step Master Mix (Applied Biosystems). The reactions use the total RNA extracted from 300 μL of plasma, with 900 nM SGAG21, 900 nM of SGAG22, and 250 nM pSGAG23 in a final volume of 30 μL. Viral RNA copies per reaction are calculated with a standard curve created by using in vitro transcribed SIVgag RNA that is serially diluted in 5 ng/μL yeast tRNA (Sigma R5636). SIV-positive plasma RNA is used as a positive control and nuclease-free water is used as a negative control. Reactions are run with the Applied Biosystems QuantStudio 6 Flex instrument (Life Technologies) using the following thermal conditions: 50° C. for 5 minutes; 95° C. for 20 seconds; [95° C. for 3 seconds, 60° C. for 30 seconds]×45 cycles. The limit of quantification for this assay is 50 copies/mL.

To detect SHIV viral RNA copies in cell pellets and tissues, a two-step RT-qPCR reaction is performed, where 2.5 μg RNA is synthesized to complementary DNA (cDNA) using 20 U Superscript II RT (Thermo Fisher/Invitrogen), 5 mM MgCl₂, 0.5 mM dNTPs, 1 mM dithithreitol (DTT), 150 ng random hexamers, 1×TaqMan PCR buffer (with 0.05% gelatin and 0.02% Tween-20), and 20 U RNAaseOut in a final volume of 30 μL. The reaction is performed with an Applied Biosystems ABI 9700 instrument using the following thermal conditions: 25° C. for 15 minutes, 42° C. for 40 minutes, 90° C. for 15 minutes, 25° C. for 30 minutes, and 5° C. hold. Following reverse transcription, qPCR is performed by adjusting the reaction to contain 1.25 U Platinum Taq (Applied Biosystems), 600 nM of SGAG21, 600 nM of SGAG22, 100 nM pSGAG23, 1× TaqMan PCR II buffer, 4.5 mM MgCl₂, and 50 nM ROX passive reference dye, in a final volume of 50 μL. Viral RNA copies per reaction are calculated with a standard curve created by using in vitro transcribed SIVgag RNA that is serially diluted in 100 ng/μL yeast tRNA (Sigma R5636). Reactions are performed in an Applied Biosystems ABI 7500 instrument using the following thermal condition: 95° C. for 2 minutes; [95° C. for 15 seconds, 60° C. for 1 minute]×45 cycles. To detect SHIV viral DNA copies in cell pellets and tissues, qPCR reactions are done using Taqman Fast Advanced Master Mix (Life Technologies). Extracted DNA is first heated at 95° C. for 5 minutes and then placed on ice. The reactions used 2.5 μg of DNA, with 600 nM of SGAG21, 600 nM of SGAG22, and 100 nM pSGAG23. Viral DNA copies per reaction are calculated with a standard curve created with linearized plasmid DNA containing the SIVgag sequence that is serially diluted in TE buffer containing 2.5 ng/mL SIV-negative rhesus genomic DNA as carrier. Reactions are run with the Applied Biosystems QuantStudio 6 Flex instrument (Life Technologies) using the following thermal conditions: 50° C. for 2 minutes; 95° C. for 20 seconds; [95° C. for 1 second, 60° C. for 20 seconds]×45 cycles. The limit of quantification for this assay is 10 copies/million cells for cell pellets and 7 copies/million cells for whole tissue biopsies.

mAb Measurement in Plasma and Tissues

Enzyme-linked immunosorbent assay (ELISA) is used to detect free mAb in plasma. Half-area 96-well Costar Assay Plates (Corning) are coated with anti-idiotype antibody at 1.5 μg/mL in carbonate-bicarbonate buffer (Thermo Fisher) and incubated overnight. Plates are washed three times with PBS-T (PBS+0.1% Tween-20) and blocked with blocking buffer (PBS+0.4% Tween-20+10% BSA) for at least 2 hours at RT. mAb concentration is calculated with a standard curve created with a serial titration of mAb diluted in blocking buffer with a range of 4.7-300 ng/m L. Heat-inactivated plasma samples are also diluted with blocking buffer. After incubating for 30 minutes at RT, plates are washed three times with 0.5 M NaCl in PBS and incubated with 1:20,000 dilution of mouse anti-human IgG₄pFc′-horseradish peroxidase (HRP) (Southern Biotech) in blocking buffer for 30 minutes at RT. Plate is washed three times with PBS-T and developed for 2 minutes using 3,3′,5,5′-tetramethylbenzidine (TMB) substrate (Southern Biotech). Reaction is stopped with 1 N H₂SO₄. Plates are read on the Synergy HTX Multi-Mode Microplate Reader (BioTek) and data are collected using software Gen5 v3.09 at two absorbance wavelengths: 650 nm for the developing reaction and 450 nm for the developed reaction after the reaction is stopped with 1 N H₂SO₄. Final OD is determined by OD_{450 nm} minus OD_{650 nm}. The limit of detection for the assay is 22.5 ng/m L. mAb in tissue is quantified in supernatants prepared from tissue homogenates by ELISA as described above. Further, the mass of the total protein in the collected tissues is determined by the Pierce Coomassie Plus Broadford Assay (Thermo Fisher) following the manufacturer's instructions. Tissue concentration of mAb is reported as the ng of mAb per mg of total protein.

Measurement of Antibody Antidrug Antibodies (ADA)

Half-area 96-well Costar Assay Plates (Corning) are coated with 2 μg/mL mAb. Plates are washed with PBS-T three times and blocked with blocking buffer for 2 hours at RT. Plates are then washed three times with PBS-T. Heat-inactivated plasma samples are serially diluted in blocking buffer, added to the plates in duplicate, and incubated at RT for 30 minutes. Plates are then washed three times with 0.5 M NaCl in PBS. To determine ADAs from RM, a secondary antibody recognizing rhesus IgG (anti-rhesus IgG1/3[1 B3]-HRP, NHP Reagent Resource) and conjugated to HRP is added. Plates are incubated at RT for 30 minutes, and then washed three times with PBS-T. TMB solution (Southern Biotech) is added at RT for 2 minutes and the reaction is stopped with 1 N H₂SO₄. Absorbance is read at 450 nm on a Synergy plate reader (BioTek). ADA titers are defined as the reciprocal of the highest dilution of the sample that yields a positive result (e.g. dilution of 1/2460=titer of 2460). A positive result is defined as twice that of background values.

Env Sequencing

Viral sequencing and analysis are adapted from previously published genome-wide SIVmac239 sequencing protocols. Viral RNA is isolated from virus stocks and plasma samples using QIAamp MinElute Virus Spin Kit following the manufacturer's instructions. Complementary DNA is generated with the SuperScript III One-Step RT-PCR with Platinum Taq (Thermo Fisher). SHIV envforward primer (GGCATAGCCTCATAAAATATCTG) and the SHIV env reverse primer (ACAGAGCGAAATGCAGTGATATT) are used to amplify a ˜4.5 kb amplicon spanning the env gene. RT-PCR reactions are performed on Eppendorf Mastercycler Pro S Thermal Cyclers using the following thermal conditions: 50° C. for 30 minutes; 94° C. for 2 minutes; [94° C. for 15 seconds, 60° C. for 1 minute, 68° C. for 4 minutes]×2 cycles; [94° C. for 15 seconds, 58° C. for 1 minute, 68° C. for 4 minutes]×2 cycles; [94° C. for 15 seconds, 60° C. for 1 minute, 68° C. for 4 minutes]×45 cycles; 68° C. for 10 minutes; and hold at 4° C. The resulting 4.9 kb fragments are purified on a 1% agarose gel and purified using NucleoSpin Gel and PCR Clean-up Kit (Macherey-Nagel). Dual-indexed Illumina MiSeq-compatible libraries are then prepared using the Nextera XT DNA Sample Prep Kit, and purified with AMPure XP magnetic beads (Beckman Coulter). Libraries are analyzed on an Agilent 2100 Bioanalyzer using the HS DNA kit (Agilent), normalized to 2 nM, pooled at an equimolar ratio, and sequenced in parallel on the Illumina MiSeq. Sequence reads are processed, where raw data are trimmed using Trimmomatic version 0.39 and aligned to the SHIVSF162 reference sequence (GenBank Accession No. KF042063.1) using BWA-MEM version 0.7.17-r1188. All bases of the alignment are evaluated and single-nucleotide polymorphisms (SNPs) and deletion/insertion polymorphisms are called for bases with a quality score above 17. Importantly, the identity of the associated read is retained for each SNP, which allowed the phase of SNPs to be considered. This information allows amino acid translations to be calculated based on the sequence of each individual read, as opposed to the consensus sequence. SNP analysis and visualization of mutations is performed using the SequenceAnalysis module, written for LabKey Server 21.3.

T Cell Assays

SHIV-specific CD8+ T cell responses in PBMC are measured by flow cytometric intracellular cytokine staining (ICS). 1×10⁶ PBMCs are incubated with overlapping 15-mer peptide pools spanning SIVmac239 Gag or Vif open reading frame and co-stimulated with CD28 and CD49d antibodies (eBiosciences) for 1 hour, followed by the incubation with Brefeldin A (Sigma-Aldrich) for an additional 8 hours. Stimulation with rhesus cytomegalovirus (RhCMV) lysate serves as a positive control while incubation without antigen serves as a background control. Cells are surface stained with antibodies for CD3, CD4, CD8, and amine-reactive dye, fixed with 2% PFA, permeabilized with BD FACS Lysing Solution (BD Biosciences), and stained intracellularly for IFN-γ, TNF-α, and CD69. Samples are collected on LSR-II instrument and FACsDIVA version 6.1 and analyzed with FlowJo v10 (Tree Star) by gating on singlet, live, CD3+, CD4−, and CD8+ cells. Responding CD8+ T cells are measured by Boolean gating on cells that are CD69+/TFN-α+ and/or CD69+/IFN-γ+.

Adoptive Transfer

To confirm sterilizing protection, cells from the infected control animals (all six animals) or uninfected mAb-treated animals (four animals in the 10 mg/kg group and all six animals in the 50 mg/kg group) are adoptively transferred into one SHIV-naïve RM per animal group. Cells are prepared an hour before infusion by resuspending in 1 mL of Hank's Buffered Salt Solution (HBSS) with 15 U/mL heparin. Recipient RM are sedated with ketamine HCl (8-20 mg/kg) or Telazol (2-5 mg/kg) and prophylactically treated with Benadryl (5 mg/kg) prior to infusion of donor cells. Donor cells are slowly infused intravenously with an infusion pump at a maximum rate of 22 mL/kg/h. Animals are monitored for at least 2 hours for post-procedural complications.

Antibodies

The following conjugated antibodies can be used in these studies: (a) from BD Biosciences, D058-1283 (CD45; PE Cy7; 1:100; cat #561294), SP34-2 (CD3; Alexa 700; 1:100; cat #557917), SP34-2 (CD3; PE; 1:20; cat #552127), LP200 (CD4; PerCP-Cy5.5; 1:40; cat #552838), RPA-T8 (CD8; PacBlu; 1:40; cat #558207), SK1 (CD8; TruRed; 1:100; cat #341051), 3 GB (CD16; Alexa 700; 1:100; cat #560713), 25723.11 (IFN-γ; APC; 1:100; cat #502512), 6.7 (TNF-α; PE; 1:100; cat #554513), 3A9 (CCR5; APC; 1:100; cat #560748), SK1 (CD8; BUV737; 1:50; cat #612754), L200 (CD4, BUV395; 1:200; cat #564107), FN50 (CD69; PE-Texas Red; 1:100; cat #562617), SP34-2 (CD3; Pacific Blue; 1:100; cat #558124); (b) from BioLegend, OKT4 (CD4; APC-Cy7; 1:100; cat #305612), RPA-T4 (CD4; APC; 1:100; cat #300537); (c) from Beckman Coulter, RM052 (CD14; PE-Texas Red; 1:40; cat #IM2707U), KC57 (HIV Gag p24; FITC; 1:100; cat #6604665); (d) from Sigma, HP-6025 (IgG4; FITC; 3:100; cat #F9890); and (e) from SouthernBiotech, HP6023 (mouse anti-human IgG4 pFc′; HRP; 1:20,000; cat #9190-05); and (f) from NHP Reagent Resource, 1 B3 (anti-rhesus IgG1/3; HRP; 1:5000). The following unconjugated antibodies can be used: 55-2F12 (SIV Gag p27; NIH AIDS Research and Reference Reagent Program), conjugated in-house to FITC using Pierce™ FITC Antibody Labeling Kit (Thermo Fisher) and used at approximately 1:100 depending on the efficacy of conjugation. Live/dead Fixable Yellow Dead Cell Stain Kit and Near-IR Dead Cell Stain Kit (Thermo Fisher) are amine-reactive dyes that can be used at 1:1000 dilution to assess cell viability.

Statistical Analyses

Time to infection is assessed by log-rank test. Differences in CCR5 expression percentages are measured by the nonparametric Kruskal—Wallis test, and differences in the number of CCR5 surface molecules are assessed by nonparametric Mann—Whitney test. Statistical significance is determined at the significant alpha level of 0.05. Statistical analyses are conducted using GraphPad Prism software version 6.0 (GraphPad Software, La Jolla, Calif.).

### Example 4—Antibody Expression Cassette

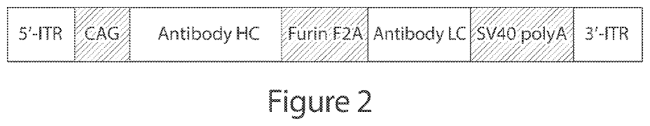

FIG. 2 shows a schematic diagram of an expression cassette for use in the present rAAV vectors encoding the present monoclonal antibodies. The cassette has the following structure: 5′ITR—CAG—Antibody Heavy Chain—Furin F2A—Antibody Light Chain—SV40 polyA-3′ITR.

These cassette components include a CMV enhancer/chicken beta-actin promoter and intron (or CAG); an SV40 polyadenylation signal (or SV40 polyA); antibody heavy and light chains; and a Furin F2A self-processing peptide cleavage site. The expression cassette is flanked by AAV serotype 2 inverted terminal repeats (ITR). In the cassette-containing bicistronic single-stranded AAV (ssAAV) vector, both the heavy and light chains are expressed from one open reading frame using a F2A self-processing peptide from FMD. The furin cleavage sequence “RKRR” for the cellular protease furin is added for removal of amino acids left on the heavy chain C-terminus following F2A self-processing. In one embodiment of this invention, the present rAAV vectors possess introns, and in another embodiment, they do not. Abbreviations: CMV, cytomegalovirus; SV40, simian virus 40; and FMD, foot-in-mouth disease virus.

### Example 5—rAAV Production

The present rAAVs can be produced according to known methods. For instance, in one such method, HEK-293 cells are transfected with a select rAAV vector plasmid and two helper plasm ids to allow generation of infectious AAV particles. After harvesting transfected cells and cell culture supernatant, rAAV is purified by three sequential CsCl centrifugation steps. Vector genome number is assessed by Real-Time PCR, and the purity of the preparation is verified by electron microscopy and silver-stained SDS-PAGE. (Mueller, et al.)

### REFERENCES

P. Maddon, et al., U.S. Pat. No. 6,451,313.
W. Dall'Acqua, et al., U.S. Pat. No. 7,083,784.
W. Olson, et al., U.S. Pat. No. 7,122,185.
L. Presta, et al., U.S. Pat. No. 7,332,581.
V. M. Litwin, et al., U.S. Pat. No. 7,345,153.
R. S. Mclvor, et al., U.S. Pat. No. 9,827,295.
P. Hotez, et al., U.S. Patent Application No. 20160376321.
D. Ballon, et al., U.S. Patent Publication No. 20170067028.
G. Buchliss, et al., U.S. Patent Publication No. 20190038724.
J. Zhou, et al., U.S. Patent Publication No. 20190078099.
M. Gasmi, et al., U.S. Patent Publication No. 20190160187.
J. A. Bluestone, et al., International Publication No. WO/1994/028027.
S. A. Morgan, et al., International Publication No. WO/1994/029351.
R. J. Owens, et al., International Publication No. WO/1995/026403.
P. J. Carter, et al., International Publication No. WO/1996/027011.
G. A. Lazar, et al., International Publication No. WO/2004/029207.
R. P. Rother, et al., International Publication No. WO/2005/007809.
A. Chamberlain, et al., International Publication No. WO/2009/086320.
T. A. Stadheim, et al., International Publication No. WO/2011/149999.
H. Zhou, International Publication No. WO/2017/079369.
Adeno-Associated Virus (AAV) Guide, Addgene Catalog (https://www.addgene. org/viral-vectors/aav/aav-guide/).
Amicus, Thermo Fisher's Brammer Bio Partner on Gene Therapy Manufacturing, Genetic Engineering & Biotechnology News, Jul. 2, 2019.
T. M. Antalis, et al., Membrane-anchored serine proteases in health and disease, Progress in Molecular Biology and Translational Science, Vol. 99 (2011).
LB. Avery, et al., (2016) Utility of a human FoRn transgenic mouse model in drug discovery for early assessment and prediction of human pharmacokinetics of monoclonal antibodies, mAbs, 8; 6, 1064-1078 DOI: 10.1080/19420862.2016, 1193660.
M. Bolles, et al., A double inactivated severe acute respiratory syndrome coronavirus vaccine provides incomplete protection in mice and induces increased eosinophilic proinflammatory pulmonary response upon challenge, J. of Virology, December 2011, 12201-12215.
D. A. Brindley, et al., Emerging Platform Bioprocesses for Viral Vectors and Gene Therapies, Bioprocess International, Apr. 18, 2016.
U. Brinkmann and R. E. Kontermann, The making of bispecific antibodies, mAbs, Vol. 9, 2:182-212 (2017).
T. H. Bugge, et al., Type II transmembrane serine proteases, J. Biol. Chem., 284(35): 23177-23181 (2009).
D. R. Burton and L. M. Walker, Rational Vaccine Design in the Time of COVID-19, Cell Host & Microbe, 27:695-698, May 13, 2020.
E. Callaway, The Race for Coronavirus Vaccines, Nature 580:576-77 (Apr. 30, 2020).
J. R. Cantor, et al., Therapeutic enzyme deimmunization by combinatorial T-cell epitope removal using neutral drift, Proc Natl Acad Sci USA, 2011 Jan. 25; 108(4): 1272-1277.
CDC, Preexposure Prophylaxis for the Prevention of HIV Infection in the United States—2021 Update Clinical Practice Guideline.
X. L. Chang, et al., Antibody-based CCR5 blockade protects Macaques from mucosal SHIV transmission, Nature Communications, volume 12, Article number: 3343 (2021).
W. H. Chen, et al., The SARS-CoV-2 Vaccine Pipeline: an Overview, Curr. Tropical Med. Reports, Springer Nature Switzerland AG (2020).
J. R. Chevillet, et al., Identification and characterization of small-molecule inhibitors of hepsin, Mol. Cancer Ther. 2008 October; 7(10): 3343-3351.
F. Chiappelli, 2019-nCoV— Toward a 4¹h Generation Vaccine, Bioinformation 16(2):139-144 (2020).
M. L. Chiu and G. L. Gilliland, Engineering antibody therapeutics, Current Opinion in Structural Biology 2016, 38:163-173.
S. Y. Choi, et al., Type II transmembrane serine proteases in cancer and viral infections, Trends in Mol. Med. 15(7): 303-312 (2009).
R. Chou, et al., Preexposure Prophylaxis for the Prevention of HIV Infection: Evidence Report and Systematic Review for the US Preventive Services Task Force, JAMA. 2019; 321(22):2214-2230.
T.-W. Chun, et al., Durable Control of HIV Infection in the Absence of Antiretroviral Therapy: Opportunities and Obstacles, JAMA. 2019; 322(1): 27-28.
N. E. Clarke and A. J. Turner, Angiotensin-Converting Enzyme 2: The First Decade, Intl. J. of Hypertension, Volume 2012, Article ID 307315, pp. 1-12.
D. Clayton, et al., Structural determinants for binding to angiotensin converting enzyme 2 (ACE2) and angiotensin receptors 1 and 2, Front. Pharmacol., 30 Jan. 2015.
C. M. Coleman, et al., Purified coronavirus spike protein nanoparticles induce coronavirus neutralizing antibodies in mice, Vaccine 32 (2014) 3169-3174.
B. Coutard, et al., The spike glycoprotein of the new coronavirus 2019-nCoV contains a furin-like cleavage site absent in CoV of the same clade, Antiviral Research 176 (2020) 104742.
M. C. Crank, et al., A proof of concept for structure-based vaccine design targeting RSV in humans, Science 365, 505-509 (2019).
S. Daya and K. I. Berns, Gene Therapy Using Adeno-Associated Virus Vectors, Clinical Microbiology Reviews, October 2008, Vol. 21, No. 4, p. 583-593.
C. E. Deal and A. B. Balazs, Vectored Antibody Gene Delivery for the Prevention or Treatment of HIV Infection, Curr Opin HIV AIDS. 2015 May; 10(3): 190-197.
M. S. Diamond and T. C. Pierson, The Challenges of Vaccine Development against a New Virus during a Pandemic, Cell Host & Microbe, 27, May 13, 2020.
J. B. Domachowske, et al., Safety, tolerability and pharmacokinetics of MED18897, an extended half-life single-dose respiratory syncytial virus perfusion F-targeting monoclonal antibody administered as a single dose to healthy preterm infants, The Pediatric Infectious Disease Journal, Vol. 37, No. 9, 886-892 (September 2018).
M. Donoghue, et al., A Novel Angiotensin-Converting Enzyme-Related Carboxypeptidase (ACE2) Converts Angiotensin Ito Angiotensin 1-9, Circulation Res., Sep. 1, 2000.
L. M. Drouin and M. Agbandje-McKenna, Adeno-associated virus structural biology as a tool in vector development, Future Virol. 2013 December; 8(12): 1183-1199.
C. Dumet, et al., Insights into the IgG heavy chain engineering patent landscape as applied to IgG4 antibody development, mAbs, Vol. 11, 8:1341-1350 (2019).
S. P. Fuchs, et al., Recombinant AAV Vectors for Enhanced Expression of Authentic IgG, PLOS One|DOI:10.1371/journal.pone.0158009, pp. 1-19, Jun. 22, 2016.
S. P. Fuchs, et al., Liver-directed but not muscle-directed AAV-antibody gene transfer limits humoral immune responses in rhesus monkeys, Mol. Therapy: Methods & Clin. Dev., 16:94-102 (March 2020).
M. R. Gardner, AAV-delivered eCD4-Ig protects rhesus macaques from high-dose SIVmac239 challenges, Sci. Transl. Med. 11, eaau5409 (Jul. 24, 2019).
M. R. Gardner, et al., Anti-Drug Antibody Responses Impair Prophylaxis Mediated by AAV-Delivered HIV-1 Broadly Neutralizing Antibodies, Molecular Therapy, Vol. 27, No. 3, 650-660 (March 2019).
M. Godar, et al., Therapeutic bispecific antibody formats: A patent applications review (1994-2017), Expert Opinion on Therapeutic Patents, Vol. 28, 3:251-276 (2018).
Y-R Guo, et al., The origin, transmission and clinical therapies on coronavirus disease 2019 (COVID-19) outbreak—an update on the status, Military Medical Res. (2020) 7:11.
J. L. Guy, et al., Identification of critical active-site residues in angiotensin-converting enzyme 2 (ACE2) by site-directed mutagenesis, FEBS Journal, 272 (2005) 3512-3520.
N. Halama, et al., Tumoral Immune Cell Exploitation in Colorectal Cancer Metastases Can Be Targeted Effectively by Anti-CCR5 Therapy in Cancer Patients, 2016, Cancer Cell 29, 587-601.
I. Hamming, et al., Tissue distribution of ACE2 protein, the functional receptor for SARS coronavirus. A first step in understanding SARS pathogenesis, J. of Pathology, 2004,
M. Hoffmann, et al., SARS-CoV-2 cell entry depends on ACE2 and TMPRSS2 and is blocked by a clinically proven protease inhibitor, Cell, 181:1-10 (2020).
M. Hoffmann, et al., A Multibasic Cleavage Site in the Spike Protein of SARS-CoV-2 Is Essential for Infection of Human Lung Cells, Molecular Cell, 78:1-6 (2020).
K. Hollevoet and P. J. Declerck, State of play and clinical prospects of antibody gene transfer, J Transl Med (2017) 15:131.
D. Hu, et al., Effective Optimization of Antibody Affinity by Phage Display Integrated with High-Throughput DNA Synthesis and Sequencing Technologies, PLOS One|DOI:10.1371/journal.pone.0129125 Jun. 5, 2015.
C. J. Hutchings, A review of antibody-based therapeutics targeting G protein-coupled receptors: an update, Expert Opinion on Biological Therapy, 1744-7682 (online) (Apr. 8, 2020).
R. Jefferys, HIV vaccine update: the “Miami macaque” as proof of-concept breakthrough? i-base, Jan. 22, 2018. (http://i-base.info/htb/date/2018/01/22).
S. Jiang, et al., SARS Vaccine Development, Emerging Infectious Diseases, 11(7): 1016-1020 (2005).
S. Jiang, et al., Roadmap to developing a recombinant coronavirus S protein receptor-binding domain vaccine for severe acute respiratory syndrome, Expert Review of Vaccines, 11(12); 1405-1413 (2012).
S. Jiang, et al., An emerging coronavirus causing pneumonia outbreak in Wuhan, China: calling for developing therapeutic and prophylactic strategies, Emerging Microbes & Infections, 9:275-277 (2020).
B. Ju, et al., Potent human neutralizing antibodies elicited by SARS-CoV-2 infection, bioRxiv doi: https://doi.org/10.1101/2020.03.21.990770.
J. Kaiser, Boys with a rare muscle disease are breathing on their own, thanks to gene therapy, May 2, 2019, Science.
A. V. Kamath, Translational pharmacokinetics and pharmacodynamics of monoclonal antibodies, Drug Discov Today: Technol (2016), doi: http://dx.doi.org/10.1016/j.ddtec.2016.09.004.
Y. Kazama, et al., Hepsin, a putative membrane-associated serine protease, activates human factor VII and initiates a pathway of blood coagulation on the cell surface leading to thrombin formation, J. Biol. Chem., 1995, 270(1): 66-72.
A. Keener, The genetic shortcut to antibody drugs, Nature 564, S16-S17 (2018).
B. Kelley, Developing therapeutic monoclonal antibodies at pandemic pace, Nature Biotechnology, Apr. 21, 2020, doi: https://www.nature.com/articles/s41587-020-0512-5.
T. Kitazawa, et al., A bispecific antibody to factors IXa and X restores factor VIII hemostatic activity in a hemophilia A model, Nature Medicine, Vol. 18, No. 10, 1570-1574 (October 2012).
G. Kohler and C. Milstein, Continuous cultures of fused cells secreting antibody of predefined specificity, Nature 1975, 256:495-497.
T. Koschubs, et al., Allosteric antibody inhibition of human hepsin protease, Biochem J. (2012) 442:483-494.
M. A. Kotterman and D. V. Schaffer, Engineering adeno-associated viruses for clinical gene therapy, Nature Reviews Genetics|AOP, published online 20 May 2014; doi:10.1038/nrg3742.
Z. Ku, et al., Nasal delivery of an IgM offers broad protection from SARS-CoV-2 variants, Nature (2021). https://doi.org/10.1038/s41586-021-03673-2.
B. Lafleur, et al., Production of human or humanized antibodies in mice, Methods Mol. Biol. 2012, 901:149-159.
C. S. Lee, et al., Adenovirus-mediated gene delivery: Potential applications for gene and cell-based therapies in the new era of personalized medicine, Genes & Diseases (2017) 4, 43-63.
C.-H. Lee, et al., IgG Fc domains that bind C1q but not effector Fcγ receptors delineate the importance of complement-effector functions, Nat. Immunol. 2017 August; 18(8): 889-98.
R. A. Liberatore and D. D. Ho, The Miami Monkey: A Sunny Alternative to the Berlin Patient, Immunity Previews, Volume 50, Issue 3, P537-539, Mar. 19, 2019.
C. Li and RJ Samulski, Engineering adeno-associated virus vectors for gene therapy, Nature Reviews, 21:255-272 (April 2020).
F. Li, et al., Structure of SARS coronavirus spike receptor-binding domain complexed with receptor, Science, 309:1864-1868 (2005).
F. Li, Receptor recognition and cross-species infections of SARS coronavirus, Antiviral Res., October 2013, 100(1).
W. Li, et al., Receptor and viral determinants of SARS-coronavirus adaptation to human ACE2, The EMBO J., (2005) 24:1634-1643.
C. C. Lim, et al., Cognizance of Molecular Methods for the Generation of Mutagenic Phage Display Antibody Libraries for Affinity Maturation, Int. J. Mol. Sci., 2019 April; 20(8): 1861.
N. Lurie, et al., Developing Covid-19 Vaccines at Pandemic Speed, N. Engl. J. Med., Perspective (April 2020).
J. M. Martinez-Navio, et al., Adeno-Associated Virus Delivery of Anti-HIV Monoclonal Antibodies Can Drive Long-Term Virologic Suppression, Immunity, 50:567-575 (2019).
J. M. Martinez-Navio, et al., Long-Term Delivery of an Anti-SIV Monoclonal Antibody With AAV, Frontiers in Immunology, March 2020, Vol. 11, Article 449.
S. Matsuyama, et al., Enhanced isolation of SARS-CoV-2 by TMPRSS2-expressing cells, PNAS, Mar. 31, 2020 117(13): 7001-7003.
K. McKeage, Ravulizumab: First Global Approval, Drugs (2019), 79:347-52.
T. Meng, et al., The insert sequence in SARS-CoV-2 enhances spike protein cleavage by TMPRSS, bioRxiv doi: https://www.biorxiv.org/content/10.1101/2020.02.08.926006v3.
J. K. Millet and G. R. Whittaker, Host cell proteases: critical determinants of coronavirus tropism and pathogenesis, Virus Res. 202 (2015) 120-134.
C. Mueller, et al., (2012). Production and discovery of novel recombinant adeno-associated viral vectors. Curr. Protoc. Microbiol. Chapter 14, Unit 14D.1.
S. Nagataa and I. Pastanb, Removal of B cell epitopes as a practical approach for reducing the immunogenicity of foreign protein-based therapeutics, Adv Drug Deliv Rev. 2009 Sep. 30; 61(11): 977-985.
M. F. Naso, Adeno-Associated Virus (AAV) as a Vector for Gene Therapy, BioDrugs (2017) 31:317-334.
D. S. Ojala, et al., Adeno-Associated Virus Vectors and Neurological Gene Therapy, The Neuroscientist, Feb. 20, 2014.
L. C. Paoletti and RC Kennedy, Neutralizing antibody induced in mice by novel glycoconjugates of Human Immunodeficiency Virus Type 1 gp120 and env2-3, J. of Infectious Diseases, 2002; 186:1597-1602.
A. Paoloni-Giacobino, et al., Cloning of the TEMPRSS2 gene, which encodes a novel serine protease with transmembrane, LDLRA, and SRCR domains and maps to 21q22.3, Genomics 44:309-320 (1997).
A. B. Patel and A. Verma, COVID-19 and angiotensin-converting enzyme inhibitors and angiotensin receptor blockers: What is the evidence? JAMA, Mar. 24, 2020.
A. Pena, Gene Therapy for Hemophilia A, SB-525, Showing Continued Benefits in Trial Data Update, Hemophelia News Today, Jun. 26, 2019.
A. Philippidis, Virus Supply Vexes Gene Therapy Developers, CMOs, Genetic Engineering & Biotechnology News, Dec. 14, 2017.
M. Poglitsch, et al., Recombinant expression and characterization of human and murine ACE2: Species-specific activation of the alternative renin-angiotensin-system, Intl. J. of Hypertension, Volume 2012, Article ID 428950, pp. 1-8.
G. Proetzel and D. C. Roopenian, Humanized FcRn mouse models for evaluating pharmacokinetics of human IgG antibodies, Methods 65 (2014) 148-153.
T. R. D. J. Radstake, et al., Formation of antibodies against infliximab and adalimumab strongly correlates with functional drug levels and clinical responses in rheumatoid arthritis, Ann Rheum Dis 2009; 68:1739-1745.
J. Riddell IV, et al., HIV Preexposure Prophylaxis: A Review, JAMA. 2018; 319041261-1268.
G. J. Robbie, et al., A Novel Investigational Fc-Modified Humanized Monoclonal Antibody, Motavizumab-YTE, Has an Extended Half-Life in Healthy Adults, Antimicrobial Agents and Chemotherapy, December 2013, Vol. 57, No. 12, pp. 6147-6143.
R. P. Rother, et al., Discovery and development of the complement inhibitor eculizumab for the treatment of paroxysmal nocturnal hemoglobinuria, Nat. Biotechnol., Vol. 25, No. 11, pp. 1256-64, November 2007.
R. A. S. Santos, et al., The ACE2/Angiotensin-(1-7)/MAS Axis of the Renin-Angiotensin System: Focus on Angiotensin-(1-7), Physiol. Rev. 98:505-553 (2018).
Z. E. Sauna, et al., Evaluating and Mitigating the Immunogenicity of Therapeutic Proteins, Trends in Biotechnology, October 2018, Vol. 36, No. 10.
T. Schlothauer, et al., Novel human IgG1 and IgG4 Fc-engineered antibodies with completely abolished immune effector functions, Protein Engineering, Design & Selection, 2016, Vol. 29, No. 10, pp. 457-466.
J. Shang, et al., Structural basis of receptor recognition by SARS-CoV-2, Nature, pages 1-19, Mar. 30, 2020.
L. W. Shen, et al., TMPRSS2: a potential target for treatment of influenza virus and coronavirus infections, Biochimie 142 (2017) 1-10.
D. Sheridan, et al., Design and preclinical characterization of ALXN1210: A novel anti-C5 antibody with extended duration of action, PLOS One, Apr. 12, 2018.
A. Shimabukuro-Vornhagen, et al., Cytokine release syndrome, J. Immunother. Cancer, 2018; 6:56.
K. Shirato, et al., Middle East Respiratory Syndrome coronavirus infection mediated by the transmembrane serine protease TMPRSS2, J. of Virology, 87(23): 12552-12561 (December 2013).
A. Shulla, et al., A transmembrane serine protease is linked to the severe acute respiratory syndrome coronavirus receptor and activates virus entry, J. of Virology, 85(2): 873-882 (January 2011).
J.-P. Silva, et al., The S228P Mutation Prevents in Vivo and in Vitro IgG4n Fab-arm Exchange as Demonstrated using a Combination of Novel Quantitative Immunoassays and Physiological Matrix Preparation, J. Biol. Chem., 2015 Feb. 27; 290(9): 5462-5469.
S. K. Singh, et al., CCR5/CCL5 axis interaction promotes migratory and invasiveness of pancreatic cancer cells, Scientific Reports, Nature, (2018) 8:1323.
P. K. Smith, et al., Measurement of protein using bicinchoninic acid, Anal. Biochem. 150:76-85 (1985).
P. Sullivan, FDA approves world's most expensive drug at $2.1M, The Hill, May 24, 2019.
J. Sun, et al., COVID-19: epidemiology, evolution, and cross-disciplinary perspectives, Trends in Mol. Med., 2020, doi: http://www.cell.com/trends/molecular-medicine/retrieve/pii/S1471491420300654?_returnURL=https %3A%2F%2 Flinkinghub.elsevier.com %2Fretrieve %2Fpii%2FS147149142030065 4%3Fshowall%3Dtrue.
S.-H. Sun, et al., A mouse model of SARS-CoV-2 infection and pathogenesis, Cell Host and Microbe (2020), doi: https://doi.org/10.1016/j.chom/2020.05.020.
F. V. Suurs, et al., A review of bispecific antibodies and antibody constructs in oncology and clinical challenges, Pharmacology & Therapeutics 201 (2019) 103-119.
W. Tai, et al., Characterization of the receptor-binding domain (RBD) of 2019 novel coronavirus: implication for development of RBD protein as a viral attachment inhibitor and vaccine, Cellular & Mol. Immun., Mar. 19, 2020.
S. H. Tam, et al., Functional, Biophysical, and Structural Characterization of Human IgG1 and IgG4 Fc Variants with Ablated Immune Functionality, Antibodies 2017, 6, 12.
P. Tamamis and C. A. Floudas, Elucidating a Key Anti-HIV-1 and Cancer-Associated Axis: The Structure of CCL5 (Rantes) in Complex with CCR5, Scientific Reports, Nature, (2014) 4:5447.
X. Tian, et al., Potent binding of 2019 novel coronavirus spike protein by a SARS coronavirus-specific human monoclonal antibody, Emerging Microbes & Infections, 9:382-385 (2020).
S. R. Tipnis, et al., A Human Homolog of Angiotensin-converting Enzyme, J. Biol. Chem., 2000 Oct. 27; 275(43): 33238-43.
P. L. Toutain and A. Bousquet-Mélou, Plasma terminal half-life, J. Vet. Pharmacol. Therap., 27:427-39.
A. J. Turner, et al., ACE2: from vasopeptidase to SARS virus receptor, Trends in Pharm. Sci, 25(6): 291-294 (2004).
M. Vaduganathan, et al., Renin—angiotensin—aldosterone system inhibitors in patients with Covid-19, N. Engl. J. Med., Special Report (April 2020).
O. Vafa, et al., An engineered Fc variant of an IgG eliminates all immune effector functions via structural perturbations, Methods (65) (2014) 114-126.
L. Vangelista and S. Vento, The Expanding Therapeutic Perspective of CCR5 Blockade, Front Immunol. 2017; 8:1981.
C. Vickers, et al., Hydrolysis of Biological Peptides by Human Angiotensin-converting Enzyme-related Carboxypeptidase, J. Biol. Chem., 2002 Apr. 26; 277(17): 14838-43.
Viral Vectors, Gene Therapy Net (http://www.genetherapynet.com/viral-vectors.html).
A. C. Walls, et al., Structure, function and antigenicity of the SARS-CoV-2 spike glycoprotein, bioRxiv doi: https://doi.org/10.1101/2020.02.19.956581.
Y. Wan, et al., Molecular Mechanism for Antibody-Dependent Enhancement of Coronavirus Entry, J. of Virology, Vol. 94, Issue 5, e02015-19 (March 2020).
N. Wang, et al., Subunit Vaccines Against Emerging Pathogenic Human Coronaviruses, Frontiers in Microbiology, 11: 298 (2020).
J. P. Wong, et al., Current ad future developments in the treatment of virus-induced hypercytokinemia, Future Med. Chem. (2017) 9(2), 169-178.
S. K. Wong, et al., A 193-amino acid fragment of the SARS Coronavirus S Protein efficiently binds Angiotensin-converting Enzyme 2, J. Biol. Chem., 279(5): 3197-3201
D. Wrapp, et al., Cryo-EM structure of the 2019-nCoV spike in the prefusion conformation, Science, 367, 1260-1263 (2020).
D. Wrapp, et al., Structural Basis for Potent Neutralization of Betacoronaviruses by Single-Domain Camelid Antibodies, Cell 181:1-12 (May 28, 2020).
Y. Wu, et al., A non-competing pair of human neutralizing antibodies block COVID-19 virus binding to its receptor ACE2, Science, 10.1126/Science.abc2241 (2020).
S. Xia, et al., Fusion mechanism of 2019-nCoV and fusion inhibitors targeting HR1 domain in spike protein, Cellular & Mol. Immunol., February 2020.
J. A. Xuan, et al., Antibodies neutralizing hepsin protease activity do not impact cell growth but inhibit invasion of prostate and ovarian tumor cells in culture, Cancer Res. 2006, 66(7): 3611-3619.
X. Yang, et al., Comprehensive Analysis of the Therapeutic IgG4 Antibody Pembrolizumab: Hinge Modification Blocks Half Molecule Exchange In Vitro and In Vivo, J Pharm Sci, 104:4002-4014, Aug. 26, 2015, https://doi.org/10.1002/jps.24620.
H. Yusuf, et al., HIV Preexposure Prophylaxis Among Adolescents in the US, JAMA Pediatr. 2020; 174(11):1102-1108.
J. Zalevsky, et al., Enhanced antibody half-life improves in vivo activity, Nat. Biotechnol. 2010 February; 28(2):157-159.
H. Zhang, et al., Angiotensin-converting enzyme 2 (ACE2) as a SARS-CoV-2 receptor: molecular mechanisms and potential therapeutic target, Intensive Care Medicine, 46:586-590 (2020).
Zhou, ACE2 and TMPRSS2 are expressed on the human ocular surface, suggesting susceptibility to SARS-CoV-2 infection, bioRxiv, doi: https://www.biorxiv.org/content/10.1101/2020.05.09.086165v1.
Q. Zhu, et al., A highly potent extended half-life antibody as a potential RSV vaccine surrogate for all infants, Sci. Transl. Med. 9, eaaj1928 (May 3, 2017).
P. Zmora, et al., TMPRSS2 isoform 1 activates respiratory viruses and is expressed in viral target cells, PLOS One Sep. 17, 2015.
A. Zumla, et al., Coronaviruses—drug discovery and therapeutic options, Nature Reviews: Drug Discovery, Vol. 15, May 2016, 327-347.

1. A humanized monoclonal antibody having the light chain variable region amino acid sequence set forth in FIG. 1A and the heavy chain variable region amino acid sequence set forth in FIG. 1B or 1C, wherein the antibody comprises a heavy chain modification that increases the antibody's terminal half-life.

2. A humanized IgG4 monoclonal antibody having the light chain variable region amino acid sequence set forth in FIG. 1A and the heavy chain variable region amino acid sequence set forth in FIG. 1B or 1C, wherein the antibody comprises (i) a heavy chain modification that inhibits half antibody formation, and (ii) a heavy chain modification that increases the antibody's terminal half-life.

3. The monoclonal antibody of claim 2, wherein the half antibody formation-inhibiting mutation is selected from the group consisting of S228P, the S228P/R409K combination, and the S228P/K447del combination (with numbering according to the EU Index).

4. The monoclonal antibody of claim 1, wherein the antibody comprises a terminal half-life-extending mutation combination selected from the group consisting of M252Y/S254T/T256E (YTE) and M428L/N434S (LS) (with numbering according to the EU Index).

5. A humanized monoclonal antibody having the light chain variable region amino acid sequence set forth in FIG. 1A and the heavy chain variable region amino acid sequence set forth in FIG. 1B or 1C, wherein the antibody comprises (i) a heavy chain modification that increases the antibody's terminal half-life, and (ii) a heavy chain modification that lowers the antibody's effector function.

6. A humanized IgG4 monoclonal antibody having the light chain variable region amino acid sequence set forth in FIG. 1A and the heavy chain variable region amino acid sequence set forth in FIG. 1B or 1C, wherein the antibody comprises (i) a heavy chain modification that inhibits half antibody formation, (ii) a heavy chain modification that increases the antibody's terminal half-life, and (iii) a heavy chain modification that lowers the antibody's effector function.

7. The monoclonal antibody of claim 6, wherein the half antibody formation-inhibiting mutation is selected from the group consisting of S228P, the S228P/R409K combination, and the S228P/K447del combination (with numbering according to the EU Index).

8. The monoclonal antibody of claim 5, wherein the antibody comprises a terminal half-life-extending mutation combination selected from the group consisting of M252Y/S254T/T256E (YTE) and M428L/N434S (LS) (with numbering according to the EU Index).

9. The monoclonal antibody of claim 5, wherein the antibody comprises an effector function-lowering mutation selected from the group consisting of L235E, L235A, F234A, G237A, D265A, an L328 substitution, A330R, F243L, the F243A/V264A combination, the E233P/F234A/L235A/G236del/G237A combination, and the S228P/L235E combination (with numbering according to the EU Index).

10. A humanized IgG2/IgG4 monoclonal fusion antibody having the light chain variable region amino acid sequence set forth in FIG. 1A and the heavy chain variable region amino acid sequence set forth in FIG. 1B or 1C, wherein (i) in the antibody, IgG2 (up to T260) is joined to IgG4 (with numbering according to the EU Index), and (ii) the antibody comprises a heavy chain modification that increases the antibody's terminal half-life.

11. The monoclonal antibody of claim 10, wherein the antibody comprises a terminal half-life-extending mutation combination selected from the group consisting of M252Y/S254T/T256E (YTE) and M428L/N434S (LS) (with numbering according to the EU Index).

12. An isolated nucleic acid molecule encoding one or more chains of the monoclonal antibody of claim 1.

13. A recombinant vector comprising the nucleotide sequence of the nucleic acid molecule of claim 12 operably linked to a promoter of RNA transcription.

14. A composition comprising (i) the monoclonal antibody of claim 1, and (ii) a pharmaceutically acceptable carrier.

15. A recombinant AAV vector comprising a nucleic acid sequence encoding the heavy chain and/or the light chain of the monoclonal antibody of claim 1.

16. The recombinant AAV vector of claim 15, wherein the nucleic acid sequence encodes the heavy chain and the light chain of the monoclonal antibody of claim 1.

17. A recombinant AAV particle comprising the recombinant AAV vector of claim 15 and an AAV capsid protein.

18. A composition comprising (i) a plurality of the AAV particles of claim 17 and (ii) a pharmaceutically acceptable carrier.

19. A method for reducing the likelihood of a human subject's becoming infected with HIV-1 comprising administering to the subject a prophylactically effective amount of the monoclonal antibody of claim 1.

20. The method of claim 19, wherein the method comprises administering the monoclonal antibody according to a PrEP regimen.

21. A method for reducing the likelihood of a human subject's becoming infected with HIV-1 comprising administering to the subject a prophylactically effective number of the AAV particles of claim 17.

22. The method of claim 19, wherein the subject has been exposed to HIV-1.

23. A method for treating a human subject who is infected with HIV-1 comprising administering to the subject a therapeutically effective amount of the monoclonal antibody of claim 1.

24. A method for treating a human subject who is infected with HIV-1 comprising administering to the subject a therapeutically effective number of the AAV particles of claim 17.

25. The method of claim 23, wherein the subject is symptomatic of an HIV-1 infection.

26. A method for treating a human subject who is infected with SARS-CoV-2 comprising administering to the subject a therapeutically effective amount of the monoclonal antibody of claim 1.

27. A method for treating a human subject who is infected with SARS-CoV-2 comprising administering to the subject a therapeutically effective number of the AAV particles of claim 17.

28. The method of claim 26, wherein the subject is symptomatic of a SARS-CoV-2 infection.

29. The method of claim 26, wherein the subject is asymptomatic of a SARS-CoV-2 infection.

30. A method for reducing the likelihood of a human subject's becoming afflicted with a CCR5-mediated disorder comprising administering to the subject a prophylactically effective amount of the monoclonal antibody of claim 1.

31. A method for treating a human subject who is afflicted with a CCR5-mediated disorder comprising administering to the subject a therapeutically effective amount of the monoclonal antibody of claim 1.

32. A method for reducing the likelihood of a human subject's becoming afflicted with a CCR5-mediated disorder comprising administering to the subject a prophylactically effective number of the AAV particles of claim 17.

33. A method for treating a human subject who is afflicted with a CCR5-mediated disorder comprising administering to the subject a therapeutically effective number of the AAV particles of claim 17.

34. The method of claim 30, wherein the CCR5-mediated disorder is selected from the group consisting of hypercytokinemia, cytokine release syndrome, Alzheimer's disease, cancer, atherosclerosis, arthritis, inflammatory bowel disease, multiple sclerosis, graft-vs-host disease (GvHD) and non-alcoholic steatohepatitis (NASH).

35. The method of claim 34, wherein the CCR5-mediated disorder is metastatic breast cancer.

36. A method for treating a human subject afflicted with a disorder comprising (i) administering to the subject a therapeutically effective amount of an agent for treating the disorder and (ii) administering to the subject a prophylactically effective amount of the monoclonal antibody of claim 1 in conjunction with step (i), wherein the agent is known to cause cytokine release syndrome.

37. A method for treating a human subject afflicted with a disorder comprising (i) administering to the subject a therapeutically effective amount of an agent for treating the disorder and (ii) administering to the subject a prophylactically effective number of the AAV particles of claim 17 in conjunction with step (i), wherein the agent is known to cause cytokine release syndrome.

38. The method of claim 36, wherein the disorder is selected from the group consisting of non-Hodgkin's lymphoma (NHL), chronic lymphocytic leukemia (CLL), and rheumatoid arthritis, and the agent is Rituxan® (rituximab).

39. The method of claim 36, wherein the disorder is selected from the group consisting of B-cell precursor acute lymphoblastic leukemia, diffuse large B-cell lymphoma (DLBCL) not otherwise specified, high grade B-cell lymphoma, and DLBCL arising from follicular lymphoma, and the agent is Kymriah® (tisagenlecleucel).

40. A kit comprising, in separate compartments, (a) a diluent and (b) a suspension of the monoclonal antibody of claim 1.

41. A kit comprising, in separate compartments, (a) a diluent and (b) the monoclonal antibody of claim 1 in lyophilized form.

42. A kit comprising, in separate compartments, (a) a diluent and (b) a suspension of a plurality of the recombinant AAV particles of claim 17.


SEQUENCE LISTING (12 sequences)

SEQ ID NO: 1 - DNA, 22 bp, Artificial Sequence
  Description of Artificial Sequence Synthetic primer
  modified_base (12)..(12): 6H,8H-3,4-dihydropyrimido[4,5-c] [1,2]oxazin-7- one
gtctgcgtcatntggtgcattc

SEQ ID NO: 2 - DNA, 29 bp, Artificial Sequence
  Description of Artificial Sequence Synthetic primer
  modified_base (7)..(7): 2-amino-6-methoxyaminopurine
  modified_base (22)..(22): 6H,8H-3,4-dihydropyrimido[4,5-c] [1,2]oxazin-7- one
cactagntgtctctgcactatntgttttg

SEQ ID NO: 3 - DNA, 32 bp, Artificial Sequence
  Description of Artificial Sequence Synthetic probe
  modified_base (5)..(5): 6H,8H-3,4-dihydropyrimido[4,5-c] [1,2]oxazin-7- one
  modified_base (11)..(11): 2-amino-6-methoxyaminopurine
cttcntcagtntgtttcactttctcttctgcg

SEQ ID NO: 4 - DNA, 23 bp, Artificial Sequence
  Description of Artificial Sequence Synthetic primer
ggcatagcctcataaaatatctg

SEQ ID NO: 5 - DNA, 23 bp, Artificial Sequence
  Description of Artificial Sequence Synthetic primer
acagagcgaaatgcagtgatatt

SEQ ID NO: 6 - protein, 4 aa, Unknown
  Description of Unknown furin cleavage sequence
RKRR

SEQ ID NO: 7 - DNA, 429 bp, Artificial Sequence
  Description of Artificial Sequence Synthetic polynucleotide
  CDS (12)..(404)
tctagaccaccatgaagttgcctgttaggctgttggtgctgatgttctggattcctgcttccagcagtgatattgtgatgacccaatctccactctccctgcctgtcactcctggagagccagcctccatctcttgcagatctagtcagcgccttctgagcagttatggacatacctatttacattggtacctacagaagccaggccagtctccacagctcctgatctacgaagtttccaaccgattttctggggtcccagacaggttcagtggcagtgggtcagggacagatttcacacttaagatcagtagagtggaggctgaggatgtgggagtttattactgctctcaaagtacacatgttcctctcacgttcggacaggggaccaaggtggaaataaaacgtaagtagtcttctcaactctaga

SEQ ID NO: 8 - protein, 131 aa, Artificial Sequence
  Description of Artificial Sequence Synthetic polypeptide
MKLPVRLLVLMFWIPASSSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIK

SEQ ID NO: 9 - DNA, 457 bp, Artificial Sequence
  Description of Artificial Sequence Synthetic polynucleotide
  CDS (12)..(434)
acgcgtccaccatggaatggagcggagtctttatctttctcctgtcagtaactgcaggtgtccactccgaggtgcagctggtggagtctggtggaggcttggtaaagcctggaggttcccttagactctcctgtgcagcctctggttacactttcagtaactattggatcggatgggtccgccaggctccaggcaaagggctggagtggattggcgatatctaccctggagggaactacatcaggaacaatgagaagttcaaggacaagaccaccctgtcagcagatacttccaagaacacagcctatctgcaaatgaacagcctgaaaaccgaggacacagccgtgtattactgtggaagcagcttcggtagtaactacgtgttcgcctggtttacttactggggccaagggactctggtcacagtctcctcaggtgagtccttaaaacctctaga

SEQ ID NO: 10 - protein, 141 aa, Artificial Sequence
  Description of Artificial Sequence Synthetic polypeptide
MEWSGVFIFLLSVTAGVHSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSS

SEQ ID NO: 11 - DNA, 457 bp, Artificial Sequence
  Description of Artificial Sequence Synthetic polynucleotide
  CDS (12)..(434)
tctagaccaccatggaatggagcggggtctttatctttctcctgtcagtaactgcaggtgtccactcccaggtccaactggtgcagtctggacctgatgtgaaaaagcctgggacttcaatgaagatgtcctgcaagacgtctggatacaccttcagtaactattggatcggatgggttaggcaggcgcctggacaaggccttgagtggattggagatatttaccctggagggaactatatcaggaacaatgagaagttcaaggacaagaccacactgacggcagacacatcgaccagcacggcctacatgcaacttggcagcctgagatctgaagacactgccgtctattactgtggaagcagcttcggtagtaactacgtgttcgcctggtttacttactggggccaagggactctggtcacagtctcctcaggtgagtccttaaaacctctaga

SEQ ID NO: 12 - protein, 141 aa, Artificial Sequence
  Description of Artificial Sequence Synthetic polypeptide
MEWSGVFIFLLSVTAGVHSQVQLVQSGPDVKKPGTSMKMSCKTSGYTFSNYWIGWVRQAPGQGLEWIGDIYPGGNYIRNNEKFKDKTTLTADTSTSTAYMQLGSLRSEDTAVYYCGSSFGSNYVFAWFTYWGQGTLVTVSS